(19) United States
(12) Patent Application Publication
(10) Pub. No.: US 2024/0033056 A1
(43) Pub. Date: Feb. 1, 2024

(54) STAINING AND SCANNING TEETH FOR DENTAL APPLICATIONS

(71) Applicant: ALIGN TECHNOLOGY, INC., San Jose, CA (US)

(72) Inventors: Ofer SAPHIER, Rehovot (IL); Roy FAHN, Petach Tikva (IL); Shai FARKASH, Hod Hasharon (IL)

(73) Assignee: ALIGN TECHNOLOGY, INC., San Jose, CA (US)

(21) Appl. No.: 18/360,141

(22) Filed: Jul. 27, 2023

Related U.S. Application Data
(60) Provisional application No. 63/370,071, filed on Aug. 1, 2022.

(51) Int. Cl. A61C 9/00; G06T 7/00

(52) U.S. Cl. CPC A61C 9/0053; G06T 7/0012; G06T 2207/30036

(57) ABSTRACT

A method for dental treatment may include generating an initial digital model of a patient's oral cavity and generating a first updated digital model of the patient's oral cavity, the first updated digital model including data representing stained locations on the patient's oral cavity. The method may also include comparing the initial digital model to the first updated digital model to identify locations of an oral problem.

### RELATED APPLICATIONS

This application claims the benefit under 35 U.S.C. § 119(e) of U.S. Provisional Patent Application No. 63/370,071, filed Aug. 1, 2022, and titled “STAINING AND SCANNING TEETH FOR DENTAL APPLICATIONS,” which is incorporated, in its entirety, by this reference.

### BACKGROUND

Staining of teeth has been used in order to visualize plaque buildup in teeth. However, current systems and methods for detecting plaque and other oral defects using stains are less than desirable in many ways. For example, existing staining methods are not accurate and lead to unmeasurable or nonquantitative evaluation of plaque and other oral defects. Existing plaque staining methods, for example, also have nonuniform staining characteristics across teeth and may even be nonuniform for plaque buildup on a single tooth. The existing methods also lack the ability to track improvements or deterioration in the patient's oral hygiene and health, such as by showing changes in plaque buildup or other oral defects over time.

### SUMMARY

As will be described in greater detail below, the present disclosure describes various systems and methods for using intraoral scanners and other imaging systems to more accurately identify and track defects using stains and staining techniques.

In addition, the systems and methods described herein may improve the functioning of a computing device by reducing computing resources and overhead for acquiring and processing images and models of the patient's dentition and identifying stain defects, thereby improving processing efficiency of the computing device over conventional approaches. These systems and methods may also improve the field of dental treatment by analyzing data to efficiently identify defects in patient's teeth.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows a method of using stain to diagnose and treat teeth, in accordance with some embodiments.

FIG. 2A shows an image of a tooth before staining, in accordance with some embodiments.

FIG. 2B shows an image of a stained tooth after staining, in accordance with some embodiments.

FIG. 2C shows an image of a stained tooth after treatment, in accordance with some embodiments.

FIG. 3 shows a method of using stain to detect defects in teeth, in accordance with some embodiments.

FIG. 4 shows a method of using stain to detect defects in teeth, in accordance with some embodiments.

FIG. 5 shows a method of using stain to detect defects in teeth with machine learning, in accordance with some embodiments.

FIG. 6 shows a method of using stain to detect contact between an orthodontic appliance and teeth, in accordance with some embodiments.

FIG. 7 shows a method of using stain to detect contact between an orthodontic appliance and teeth, in accordance with some embodiments.

FIGS. 8A and 8B show images of detentions and appliances at various stages of staining, in accordance with some embodiments.

FIG. 9 shows a block diagram of an example computing system capable of implementing one or more embodiments described and/or illustrated herein, in accordance with some embodiments.

FIG. 10 shows a block diagram of an example computing network capable of implementing one or more of the embodiments described and/or illustrated herein, in accordance with some embodiments.

FIG. 11 illustrates an exemplary tooth repositioning appliance or aligner that can be worn by a patient in order to achieve an incremental repositioning of individual teeth in the jaw, in accordance with some embodiments.

FIG. 12 illustrates a tooth repositioning system, in accordance with some embodiments.

FIG. 13 shows a method of orthodontic treatment using a plurality of appliances, in accordance with embodiments;

FIG. 14 shows a method for digitally planning an orthodontic treatment, in accordance with embodiments; and

FIG. 15 shows a simplified block diagram of a data processing system, in accordance with embodiments.

### DETAILED DESCRIPTION

The following detailed description provides a better understanding of the features and advantages of the improvements described in the present disclosure in accordance with the embodiments disclosed herein. Although the detailed description includes many specific embodiments, these are provided by way of example only and should not be construed as limiting the scope of the inventions disclosed herein.

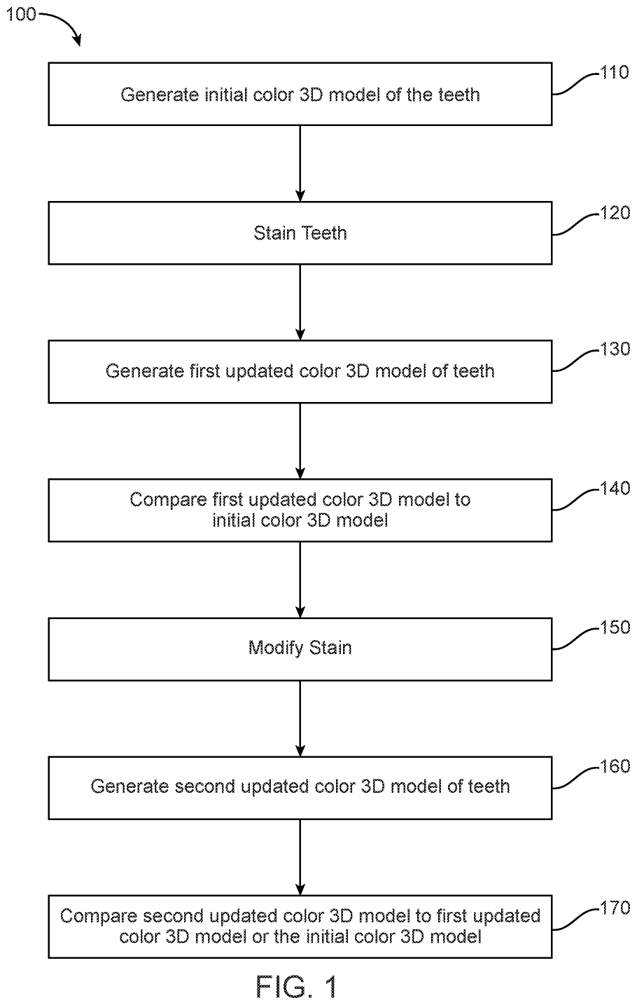

FIG. 1 shows a method 100 of using stain to diagnose and treat teeth. The method 100 may be used in many ways. For example, the method 100 may be used to evaluate a patient's brushing technique, such as to determine how well their brushing removes plaque buildup. In some embodiments, the method 100 may be used to evaluate a dental professional's ability to remove plaque. The method 100 may include imaging a patient's oral cavity, such as their dentition at block 110, applying a coloring agent to or otherwise staining a patient's teeth at block 120, generating a first updated color 3D model of the patient's teeth at block 130, comparing the first updated color 3D model to the initial color 3D model at block 140, modifying the stain, such as by brushing or scraping plaque from the patient's teeth, at block 150, generating a second updated color 3D model of the patient's teeth at block 160, and comparing the second updated color 3D model to the first updated color 3D model at block 170. Each step of the method 100 is described in more detail, herein.

At block 110 an initial color 3D model of the patient's teeth is generated. A color 3D model of a patient's teeth may be a digital representation of the patient's teeth and can include surface topography data and may be a 3D digital model of the patient's intraoral cavity (including teeth, gingival tissues, etc.), along with a color mapping, or color texture applied to the 3D digital model. The surface topography data can be generated by directly scanning the intraoral cavity using a suitable scanning device, such as a hand-held intraoral scanner.

The initial color 3D model of the patient's teeth may be a high-fidelity model generated by capturing a series of images of the patient's oral cavity and stitching the images together in order to generate a 3D model of the patient's entire oral cavity, their dentition and/or gingiva. In some embodiments, in order to accurately stitch together the images to generate the 3D model, successive images may overlap each other by at least 70%, at least 80%, or at least 90%. Overlapping the images allows for each location on a patient's tooth to be visible in at least 3, 5, or 10 frames and allows for an accurate generation of the initial 3D model of the patient's teeth. The images may include one or both of three-dimensional data related to the surface topography of the patient's teeth and color data representative of the color of the patient's teeth. During the stitching process, the color data is mapped to a particular location on the generated 3D model. Upon generation of the initial 3D model, a baseline of the surface topography and color of the patient's oral cavity, such as their dentition and gingiva is determined.

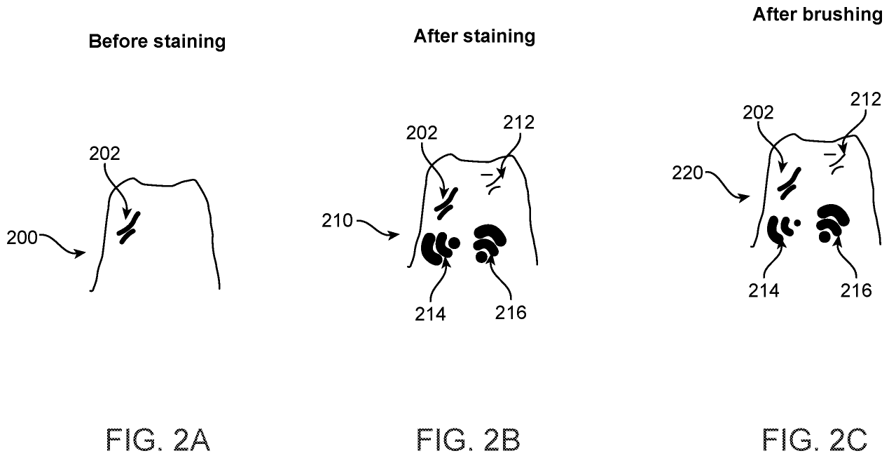

FIG. 2A depicts an image 200 of an initial color 3D model of a patient's tooth. The image 200 includes an existing color feature 202 that is present on the tooth before staining.

Referring back to FIG. 1, at block 120 the patient's teeth are stained. The stain may be a stain that attaches to defects in the patient's teeth, such as to plaque, caries, demineralized areas of the patient's teeth, or cancerous cells. The stain may come in the form of an oral tablet that is chewed and swished around in the patient's mouth. During this process the stain is absorbed by the defects in the patient's teeth, such as the plaque, caries, demineralized areas of the patient's teeth, or cancerous cells. Stains may be colored with a dye in the visible light spectrum, such as a red, blue, or purple. In some embodiments, stains may be colored with dyes visible in the near infrared or ultraviolet wavelengths of light. In some embodiments, the stains may fluoresce when exposed to certain wavelengths of light.

The stains and the stain application process may not result in an ideal or consistent application of stain to the oral problems. Oral problems described herein may include oral cavity pathologies, such as health issues with the patient's gingiva, tongue, cheeks, or teeth, such as to plaque, caries, demineralized areas of the patient's teeth, or cancerous cells. Teeth or locations of teeth with similar amounts or degrees of defects, such as plaque, caries, demineralized areas of the patient's teeth, or cancerous cells, may be stained to different degrees or magnitudes, which may result in some locations that have plaque (or other oral defects) having a first amount of stain and other locations with the same amount of plaque (or other oral defects) having a second amount of stain. In addition, magnitude of the staining from one application of stain to another application of stain may not be consistent. The inconsistency may occur even when the degree the oral problem, such as of caries, plaque, demineralization, or cancer remains the same. For example, the patient may stain their teeth twice in the same day, yet similar locations on their teeth may show different amounts of stain. Such inconsistencies in staining may lead to failure to detect oral problems, such as when the stain is to light for visual observation and/or a lack of accuracy in determining the location and degree of dental caries or plaque demineralization or other oral problems, such as cancer.

At block 130 a first updated color 3D model of the patient's teeth is generated. The updated color 3D model of a patient's teeth may be similar to the initial color 3D model and may be a digital representation of the patient's teeth and can include surface topography data, such as a 3D digital model, of the patient's intraoral cavity along with a color mapping or color texture applied to the 3D digital model. The surface topography data can be generated by directly scanning the intraoral cavity using a suitable scanning device, such as a hand-held intraoral scanner.

The updated color 3D model of the patient's teeth may be based on the initial color 3D model of the patient's teeth generated in block 110. In some embodiments, the surface topography of the initial color 3D model is used for the updated color 3D model. The color data for the updated color 3D model may be generated by capturing a series of images of the patient's oral cavity, aligning the images with the existing 3D topography data, and mapping the color data to the 3D topography data or applying the color data as a color texture to the 3D topography data to generate the updated color 3D model of the patient's teeth. In some embodiments, in order to accurately generate the updated color data for the updated color 3D model, successive images of the patient's oral cavity captured by the intraoral scanner may overlap each other by less than those of the images used to generate the initial color 3D model. For example, in some embodiments, the images may overlap by less than 30%, less than 20%, or less than 10%. The images may overlap by such low percentages because accurately aligning new images to an existing model can be accomplished with lower overlap percentages than during the initial scan. Overlapping the images in this way may result in most locations of the patient's teeth being visible in less than three images. In some embodiments, the majority of the locations of the patient's teeth may be imaged in only one image. The images may include one or both of three-dimensional data related to the surface topography of the patient's teeth and color data representative of the color of the patient's teeth. During the stitching process, the new color data is mapped to a particular location on the initial 3D model. Upon generation of the updated color 3D model, the surface topography and the stained color of the structures of the patient's oral cavity is determined.

FIG. 2B depicts an image 210 of a first updated color 3D model of the patient's tooth. The image 210 includes the existing color feature 202 that was present in the initial 3D scan and three additional color features 212, 214, 216. Color feature 212 may be a lightly stained portion of the patient's oral cavity, such as a tooth, with an oral problem wherein the oral problem absorbed a small amount of dye or staining. Color features 214, 216 may be heavily stained portions of the patient's oral cavity, such as locations with an oral problem where the oral problem absorbed a large amount of dye or staining. In some embodiments, the degree of staining may not be indicative of the degree of the oral problem. For example, a location with a high amount or density of dental plaque may be depicted with a small amount of dye or staining while a location with a similarly high amount or density of dental plaque may also be depicted with a high amount of dye or staining.

At block 140 the first updated color 3D model is compared to the initial color 3D model. The comparison shows locations of plaque identified by the staining. During the comparison, the pre-existing colored dental features within the color data of the initial 3D model are removed from the first updated color 3D model in order to remove pre-existing color, such as color that is not indicative of an oral problem. In some embodiments, color data from the initial color 3D model is subtracted from the color data in the first updated color 3D model. For example, with reference to FIG. 2B, the color feature 202 that existed in the initial color 3D data may be removed from the first updated color 3D model during the comparison. In some embodiments, a new color 3D model is generated based on the comparison.

At block 150 the teeth staining may be modified. For example, with patient hygiene training, such as in order to show a patient the effectiveness of their toothbrushing technique and to show them where and how their toothbrushing technique may be improved, a patient may brush their teeth. In some embodiments, such as for dental hygienist training to show a dental hygienist the effectiveness of their plaque removal techniques, a dental hygienist may perform a routine plaque removal or tooth cleaning process on the patient's teeth.

At block 160 a second updated color 3D model of the patient's teeth is generated. The second updated color 3D model of a patient's teeth may be similar to the first updated color 3D model and may be a digital representation of the patient's teeth and can include surface topography data, such as a 3D digital model, of the patient's intraoral cavity along with a color mapping or color texture applied to the 3D digital model. The surface topography data can be generated by directly scanning the intraoral cavity using a suitable scanning device, such as a hand-held intraoral scanner.

The second updated color 3D model of the patient's teeth may be based on the initial color 3D model of the patient's teeth generated in block 110 or the first updated color 3D model of the patient's teeth generated in block 130. In some embodiments, the surface topography of the initial color 3D model is used for the second updated color 3D model. The color data for the second updated color 3D model may be generated by capturing a series of images of the patient's oral cavity, aligning the images with the existing 3D topography data and mapping the color data to the 3D topography data or applying the color data as a color texture to the 3D topography data to generate the second updated color 3D model of the patient's teeth. As with the first updated color 3D model, in some embodiments, in order to accurately generate the updated color data for the second updated color 3D model, successive images of the patient's oral cavity captured by the intraoral scanner may overlap each other by less than those of the images used to generate the initial color 3D model. The images may include one or both of three-dimensional data related to the surface topography of the patient's teeth and color data representative of the color of the patient's teeth. During the stitching process, the new color data is mapped to a particular location on the initial 3D model or the first updated color 3D model. Upon generation of the second updated color 3D model, the surface topography and the stained color of the patient's oral cavity is determined.

FIG. 2C depicts an image 220 of a second updated color 3D model of the patient's tooth. The image 220 includes the existing color feature 202 that was present in the initial 3D scan, and the three additional color features 212, 214, 216. After the stain modification procedure, the staining of the color feature 212, the lightly stained portion of the patient's oral cavity, such as a location with an oral problem wherein the oral problem absorbed a small amount of dye, remains similar to that of the first updated color 3D digital model. Color features 214, 216, which were both heavily stained portions of the patient's oral cavity, such as locations with an oral problem where the oral problem absorbed a large amount of dye or staining, show differing amounts of dye removal. Color feature 214 shows a lower amount of staining in the second updated color 3D model while color feature 216 shows the same amount of staining in the second updated color 3D model. The differing amount may indicate that a portion of the oral problem was removed.

At block 170 a comparison of the second updated color 3D model may be made with respect to the first updated color 3D model or the initial color 3D model. The comparison may indicate how much plaque was left after brushing. For example, in some embodiments, such as when the stain is not removed before brushing or plaque removal, the comparison may be made directly between the first updated color 3D model in the second updated color 3D model. In some embodiments, such as when the stain is removed and new stain is applied before generation of the second updated color 3D model, the second updated color 3D model may be compared to the initial color 3D model in order to assess how much plaque was left after brushing or plaque removal. In some embodiments, the second updated color 3D model may be first compared to the initial color 3D model in order to remove existing color features, such as color feature 202, and then the modified second updated color 3D model may be compared to the first updated color 3D model in order to determine assess the effectiveness of brushing or plaque removal.

In some embodiments, such as when dyes that are not visible to the naked eye are used, the dye may not be removed between the first updated color 3D model and the generation of the second updated color 3D model because the patient and/or dental professional is not able to see the dye when performing the brushing, plaque removal, or other dental treatment and are therefore their actions are not affected or biased by the existence of dye visible to the naked eye.

In this way, method 100 can be used to evaluate and train patients and dental professionals in brushing, plaque removal, and other techniques.

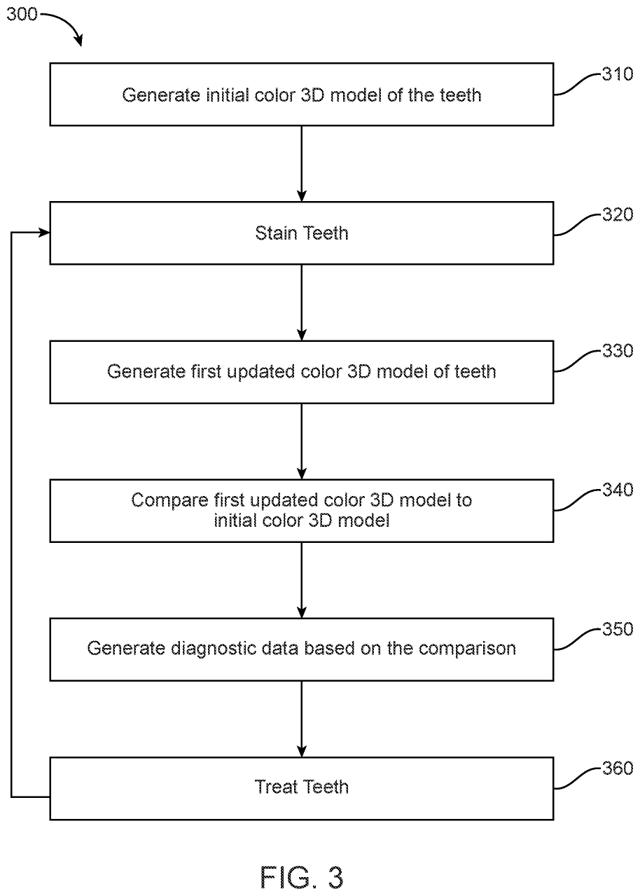

FIG. 3 shows a method 300 of using stain to track teeth health. The method 300 may be used in many ways. For example, the method 300 may be used to track the location, size, and extent of plaque, demineralization, and/or caries over time. In some embodiments, the location size and extent of plaque, demineralization, and/or caries may be determined based on scans of stained teeth during each regular dental appointment. The results of the scans may be evaluated over time to determine whether a patient's oral problems are improving or deteriorating. In some embodiments, dental intervention may be performed in response to deteriorating oral problems. The method 300 may include imaging a patient's oral cavity at block 310, applying coloring agent to or otherwise staining a patient's teeth at block 320, generating an updated color 3D model of the patient's teeth at block 330, comparing the updated color 3D model to the initial color 3D model at block 340, generating diagnostic data based on the comparison at block 350, and treating the teeth based on the diagnosis at block 360. The process may then proceed back to block 320 where block 320, 330, 340, 350, and 360 may be repeated. Each step of the method 300 is described below in more detail.

At block 310, an initial color 3D model of the patient's teeth is generated. A color 3D model of a patient's teeth may be a digital representation of the patient's teeth and can include surface topography data, such as a 3D digital model, for the patient's intraoral cavity (including teeth, gingival tissues, etc.), along with a color mapping, or color texture applied to the 3D digital model. The surface topography data can be generated by directly scanning the intraoral cavity using a suitable scanning device, such as a hand-held intraoral scanner.

The initial color 3D model of the patient's teeth may be a high-fidelity model generated by capturing a series of images of the patient's oral cavity and stitching the images together in order to generate a 3D model of the patient's entire oral cavity. In some embodiments, in order to accurately stitch together the images to generate the 3D model, successive images may overlap each other by at least 70%, at least 80%, or at least 90%. Overlapping the images allows for each location on a patient's tooth to be visible and at least 3, 5, or 10 frames and allows for an accurate generation of the initial 3D model of the patient's teeth. The images may include one or both of three-dimensional data related to the surface topography of the patient's teeth and color data representative of the color of the patient's teeth. During the stitching process, the color data is mapped to a particular location on the generated 3D model. Upon generation of the initial 3D model, a baseline of the surface topography and color of the patient's oral cavity is determined.

At block 320 the patient's teeth are stained. The stain may be a stain that attaches to defects in the patient's teeth, such as to plaque, caries, demineralized areas of the patient's teeth, or cancerous cells. The stain may come in the form of an oral tablet that is chewed and swished around in the patient's teeth. During this process the stain is absorbed by or attached to the defects in the patient's teeth, such as plaque, caries, demineralized areas of the patient's teeth, or cancerous cells. Stains may be colored with a dye in the visible light spectrum, such as a red, blue, or purple. In some embodiments, stains may be colored with dyes visible in the near infrared or ultraviolet wavelengths of light. In some embodiments, the stains may fluoresce when exposed to certain wavelengths of light.

The stains and the stain application process may not result in an ideal or uniform application of stain to the oral problems, such as caries or plaque or demineralization. Teeth or locations of teeth with similar amounts or degrees defects, such as caries, plaque, demineralization, or cancer may be stained to different degrees which may result in some locations that have plaque having a first amount of stain and other locations with the same amount of plaque having a second amount of stain. In addition, staining from one application to another may not be consistent even when the degree of caries or plaque or demineralization remains the same.

At block 330 an updated color 3D model of the patient's teeth is generated. The updated color 3D model of a patient's teeth may be similar to the initial color 3D model and may be a digital representation of the patient's teeth and can include surface topography data, such as a 3D digital model, for the patient's intraoral cavity along with a color mapping or color texture applied to the topography data. The surface topography data can be generated by directly scanning the intraoral cavity using a suitable scanning device, such as a hand-held intraoral scanner.

The updated color 3D model of the patient's teeth may be based on the initial color 3D model of the patient's teeth generated in block 310. In some embodiments, the surface topography of the initial color 3D model is used for the updated color 3D model. The color data for the updated color 3D model may be generated by capturing a series of images of the patient's oral cavity, aligning the images with the existing 3D topography data and mapping the color data to the 3D topography data or applying the color data as a color texture to the 3D topography data to generate the updated color 3D model of the patient's teeth. In some embodiments, in order to accurately generate the updated color data for the updated color 3D model, successive images of the patient's oral cavity captured by the intraoral scanner may overlap each other by less than those of the images used to generate the initial color 3D model. For example, in some embodiments, the images may overlap by less than 30%, less than 20%, or less than 10%. The images may overlap by such low percentages because accurately aligning new images to an existing model can be accomplished with lower overlap percentages than during the initial scan. Upon generation of the updated color 3D model the surface topography and the stained color of the patient's oral cavity is determined.

At block 340 the updated color 3D model is compared to the initial color 3D model. The comparison shows the extent of dental defects identified by the staining. During the comparison, the pre-existing colored dental features within the color data of the initial 3D model are removed from the updated color 3D model in order to remove color data that is not indicative of an oral problem. In some embodiments, color data from the initial color 3D model is subtracted from the color data in the updated color 3D model. In some embodiments, a new color 3D model is generated based on the comparison.

At block 350, diagnostic data based on the comparison may be generated. After revising the updated color 3D model based on the initial 3D color model, the updated color 3D model may be used to diagnose the extent of the patient's plaque, caries, demineralization, or other oral problem, such as cancer. For example, each location of an oral problem may be masked or otherwise identified on one of the color 3D models. In some embodiments, the overall surface area or volume of the oral problem may be determined based upon the updated color 3D model and tracked.

Optionally, at block 360 the patient's teeth may be treated. If the diagnostic data indicates that the patient's teeth should be treated, then the patient's teeth may be treated at block 360, such as by installing a filling, removing plaque, or other treatment processes. Whether or not the patient is treated at block 360, the process 300 may proceed to block 320 and the staining and imaging steps may be conducted again. The process of at block 320, 330, 340, 350, 360 may be repeated a plurality of times, such as during each dental visit. The diagnostic data generated at block 350 during each dental visit may then be compared over time in order to determine the effectiveness of the treatment, the patient's dental hygiene practices, etc. In some embodiments, treatment at block 360 may be carried out based on changes in the diagnostic data over time and/or the effectiveness of the dental treatment as determined by the changes in the dental data over time.

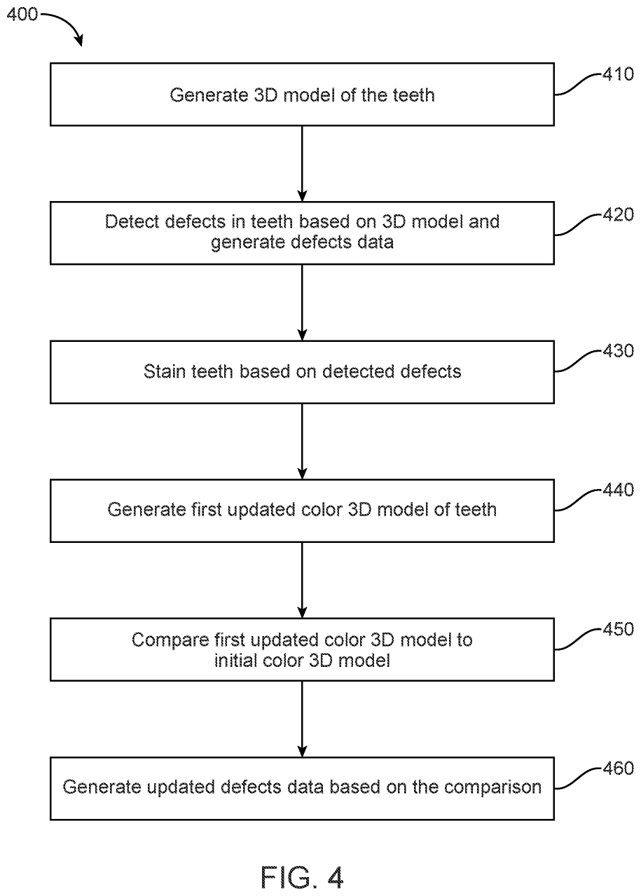

FIG. 4 shows a method 400 of using stain to detect defects in teeth. In some embodiments, an initial intraoral scan may be conducted. Based on the results of the intraoral scan, a teeth staining and scanning diagnostic procedure may be performed. For example, if the initial intraoral scan indicated the patient may have dental defect, such as dental caries, then a staining and scanning procedure may be completed. Method 400 may include generating an initial color 3D model of the teeth at block 410, detecting defects in the teeth based on the 3D model and generating defects data at block 420, staining the teeth based on the detected defects at block 430, generating an updated color 3D model of the teeth at block 440, comparing the updated color 3D model to the initial color 3D model at block 450, and generating updated defects data based on the comparison at block 460.

At block 410 an initial color 3D model of the patient's teeth is generated. A color 3D model of a patient's teeth may be a digital representation of the patient's teeth and can include surface topography data, such as a 3D digital model, for the patient's intraoral cavity (including teeth, gingival tissues, etc.), along with a color mapping, or color texture applied to the 3D digital model. The surface topography data can be generated by directly scanning the intraoral cavity using a suitable scanning device, such as a hand-held intraoral scanner.

In some embodiments, the initial color 3D model may be generated using near infrared, ultraviolet, and/or fluorescence data gathered from an intraoral 3D scanner. The near infrared, ultraviolet and/or fluorescence data may reveal oral problems not apparent to the naked eye or with exposure to visible wavelengths of light. For example, near infrared data can be used to see subsurface oral problems, such as dental caries.

The initial color 3D model of the patient's teeth may be a high-fidelity model generated by capturing a series of images of the patient's oral cavity and stitching the images together in order to generate a 3D model of the patient's entire oral cavity or the dentition, gingiva, or other oral structure. During the stitching process, the color data is mapped to a particular location on the generated 3D model.

At block 420 dental defects, such as caries, plaque, and/or demineralization, may be detected from the scan data in the initial 3D model of the teeth. The defects data may include a location and/or extent of the defect in the digital model of the patient's teeth. If dental defects are detected in the initial 3D model of the patient's teeth the process may proceed to block 430.

At block 430 the patient's teeth are stained. The stain may be a stain that attaches to defects in the patient's teeth, such as to plaque, caries, demineralized areas of the patient's teeth, or cancerous cells. The stain may come in the form of an oral tablet that is chewed and swished around in the patient's teeth. During this process, the stain is absorbed by or attaches to the defects in the patient's teeth, such as plaque, caries, demineralized areas of the patient's teeth, or cancerous cells. Stains may be colored with a dye in the visible light spectrum, such as a red, blue, or purple. In some embodiments, stains may be colored with dyes visible in the near infrared or ultraviolet wavelengths of light. In some embodiments, the stains may fluoresce when exposed to certain wavelengths of light.

At block 440 an updated color 3D model of the patient's teeth is generated. The updated color 3D model of a patient's teeth may be similar to the initial color 3D model and may be a digital representation of the patient's teeth and can include surface topography data, such as a 3D digital model, for the patient's intraoral cavity along with a color mapping or color texture applied to the 3D digital model. The surface topography data can be generated by directly scanning the intraoral cavity using a suitable scanning device, such as a hand-held intraoral scanner.

The updated color 3D model of the patient's teeth may be based on the initial color 3D model of the patient's teeth generated in block 410. In some embodiments, the surface topography of the initial color 3D model is used for the updated color 3D model. The color data for the updated color 3D model may be generated by capturing a series of images of the patient's oral cavity, aligning the images with the existing 3D topography data and mapping the color data to the 3D topography data or applying the color data as a color texture to the 3D topography data to generate the updated color 3D model of the patient's teeth.

The images may include one or both of three-dimensional data related to the surface topography of the patient's teeth and color data representative of the color of the patient's teeth. During the stitching process, the new color data is mapped to a particular location on the initial 3D model. The mapping places the color data on the digital model in a location that corresponds to the location of the color in the real world. Upon generation of the updated color 3D model the surface topography and the stained color of the patient's oral cavity is determined.

At block 450 the updated color 3D model is compared to the initial color 3D model. The comparison shows how much plaque or other oral defect was identified by the staining. During the comparison, the pre-existing colored dental features within the color data of the initial 3D model are removed from the updated color 3D model in order to remove color data that is not indicative of an oral problem. In some embodiments, color data from the initial color 3D model is subtracted from the color data in the updated color 3D model. In some embodiments, a new color 3D model is generated based on the comparison.

At block 460, diagnostic data based on the comparison may be generated. After revising the updated color 3D model based on the initial 3D color model, the updated color 3D model may be used to diagnose the extent of the patient's plaque, caries, demineralization, or other oral problem, such as cancer. For example, each location of an oral problem may be masked or otherwise identified on one of the color 3D models. In some embodiments, the overall surface area or volume of the oral problem may be determined based upon the updated color 3D model and tracked. The diagnostic data may be used in combination with the defects data generated at block 420 in order to more accurately evaluate in treatment the patient's oral problems.

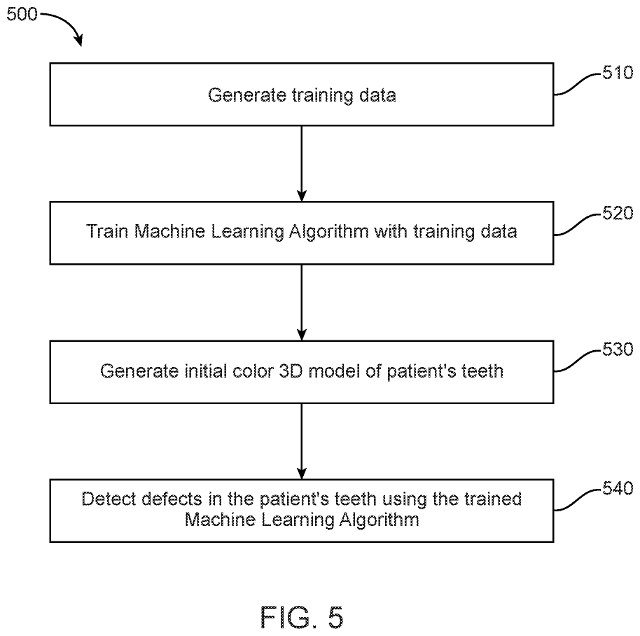

FIG. 5 shows a method 500 of using stain to detect defects in teeth with machine learning, in accordance with some embodiments. In some embodiments, staining may be used in the training of a machine learning algorithm for detecting oral problems. For example, stained teeth images may be used to tag data on unstained teeth images of the same teeth. The tagged images may be used to train a machine algorithm capable of detecting the oral problems or assessing a probability that an oral problem exists in unstained teeth images. The method 500 may include generating the training data at block 510, training algorithm the training data in block 520, generating an initial color 3D model of the patient's teeth at block 530 and detecting defects or oral problems in the patient's teeth using a trained machine learning algorithm at block 540.

At block 510 training data for training the machine learning algorithm is generated. The training data may be a combination of a plurality of clean scan data, such as scan data from the plurality of patient's unstained teeth, such as an initial color 3D models, and corresponding tagged scan data that includes tagged oral problems on the patient's teeth. Scan data of patient's stained teeth may be used as the tagged scan data. Oral problems may be automatically tagged in the scan data of patient's stained teeth using image recognition techniques or may be tagged by indicating the location of staining the patient's teeth, such as by highlighting or placing a bounding box around each defect or oral problem. In some embodiments, the tagging may be on a pixel-by-pixel basis, wherein the tagging is made for each pixel in the image or model.

In some embodiments, the training data may be clean scan data that is tagged based on the tagged scan data from color scan data of stained teeth. The clean scan data may be an initial color 3D model of the patient's teeth. A color 3D model of a patient's teeth may be a digital representation of the patient's teeth and can include surface topography data, such as a 3D digital model, for the patient's intraoral cavity (including teeth, gingival tissues, etc.), along with a color mapping or color texture applied to the 3D digital model. The surface topography data can be generated by directly scanning the intraoral cavity using a suitable scanning device, such as a hand-held intraoral scanner without stain applied to the teeth.

The tagging data may be generated by staining the patient's teeth using stain for identifying oral problems and then scanning the stained teeth. The tagging data may be generated based on a color 3D model of a patient's teeth that may be similar to the clean 3D model and may be a digital representation of the patient's teeth and can include surface topography data, such as a 3D digital model, of the patient's intraoral cavity, along with a color mapping or color texture applied to the 3D digital model. The stained areas of the teeth may be identified through image processing techniques, such as, for example, edge detection or may be manually indicated, such as using a bounding box around the stained locations. In some embodiments, the identification may be on a pixel-by-pixel basis, wherein the identification is made for each pixel in the image or model.

In some embodiments, the stained data may be compared against the clean data, as described herein. The machine learning or artificial intelligence system may be a neural network and may include one or more AI schemes, such as a convolutional neural network, deep learning, etc., and may correspond to, for example, MobileNet, EfficientNet, VGG, etc. The neural network may undergo training via training data in order to recognize the various classifications described herein. The neural network may determine categorical classifications, which may correspond to various categorical classifications as described herein.

In addition, the neural network may include a binary classifier. The binary classifier may determine the binary classifications using binary cross-entropy, which may utilize a loss function to predict a probability of between two possible values for each binary classification. The neural network may determine binary classifications, which may correspond to binary classifications described herein, such as whether or not the location has caries, plaque, demineralization, cancer, and other examples described herein.

In some embodiments, the machine learning algorithm may be a classifier, such as ML classifiers for binary classification and/or categorical classification. In some embodiments, the term “binary classification” may refer to characteristics that may be defined as having one of two states (e.g., yes or no). With respect to the scan data, examples of binary classifications may include, without limitation, whether a particular location of the tooth as a potential oral problem, such as caries, plaque, demineralization, cancer, and other examples described herein.

In some embodiments, the term “categorical classification” may refer to characteristics that may be classified into one or more categories. In some embodiments, the characteristics may be classified into one or more sets of mutually exclusive categories. With respect to the image data, examples of categorical classifications may include, without limitation, having one or more caries, plaque, demineralization, and other examples described herein.

The method may detect, from the plurality of images, scan data, or 3D models, one or locations of oral problems on the patient's oral cavity. For example, the machine learning algorithm may detect caries, plaque, demineralization, cancer, and other examples described herein.

The systems described herein may perform the detection in a variety of ways. In one example, the machine learning algorithm may use computer vision and/or object recognition as described herein. For example, the machine learning algorithm may detect oral problems in the teeth, such as dental caries, plaque, demineralization, cancer, or other oral problems, and form bounding boxes around each detected oral problem. A bounding box may define outer boundaries of an identified structure and may be rectangular and/or square in shape to simplify processing, particularly for irregularly shaped objects. In some embodiments, the identification may be on a pixel-by-pixel basis, wherein the identification is made for each pixel in the image or model.

At block 520 a machine learning algorithm is trained with the training data. For example, the clean scan data that is tagged based on the stain scan data may be used to train the machine learning algorithm to identify oral problems based on unstained or clean scan data.

Each machine learning algorithm may be trained to identify one or more types of oral problems. For example, in some embodiments, a machine learning algorithm may be trained to identify dental caries, while other machine learning algorithms are trained to identify another type of oral problem, such as demineralization, plaque, cancer and/or other oral problems.

At block 530 a color 3D model of the patient's teeth is generated. An initial color 3D model of a patient's teeth may be a digital representation of the patient's teeth and can include surface topography data, such as a 3D digital model, for the patient's intraoral cavity (including teeth, gingival tissues, etc.), along with a color mapping, or color texture applied to the 3D digital model. The surface topography data can be generated by directly scanning the intraoral cavity using a suitable scanning device, such as a hand-held intraoral scanner. In some embodiments, multiple machine learning algorithms may be trained.

At block 540 the color 3D model of the patient's teeth is processed by the machine learning algorithm that was trained at block 520. The machine learning algorithm processes the color 3D model according to its training and outputs an annotated 3D model of the patient's teeth. The annotations may indicate locations and types of oral problems identified by the algorithm. In some embodiments, the color 3D model of the patient's teeth may be processed by multiple machine learning algorithms and the results of the processing may be applied or otherwise combined into a single annotated color 3D model of the patient's teeth.

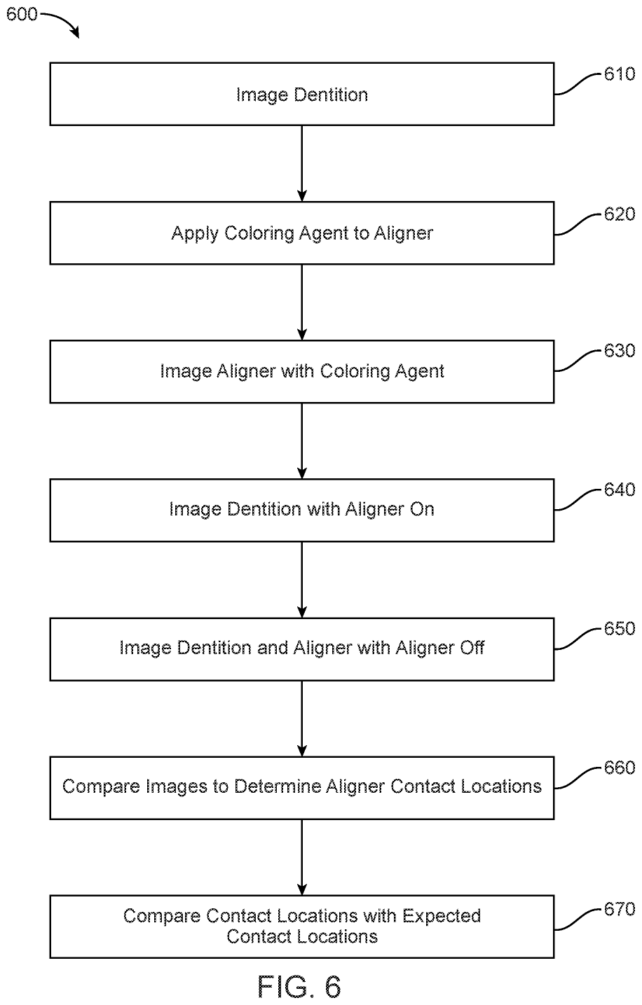

FIG. 6 shows a method 600 of using stain to detect contact between an orthodontic appliance, such as an orthodontic aligner or a retainer and teeth, in accordance with some embodiments. In some embodiments, stain may be added to an aligner or retainer before the appliance is placed on and then removed from the patient's teeth in order to determine contact between the appliance and the patient's teeth.

At block 610 in the initial scan of the patient's teeth is generated. An initial scan of the patient's teeth may include generating a color 3D model of a patient's teeth that may be a digital representation of the patient's teeth and can include surface topography data, such as a 3D digital model, for the patient's intraoral cavity (including teeth, gingival tissues, etc.), along with a color mapping, or color texture applied to the 3D digital model. The surface topography data can be generated by directly scanning the intraoral cavity using a suitable scanning device, such as a hand-held intraoral scanner.

At block 620 colorant or a coloring agent such as a stain, dye, or color paste may be added to the internal surfaces of an appliance cavity. The colorant may be evenly applied to a dental appliance formed based on a three-dimensional scan of the patient's teeth. In some embodiments, the dental appliance may be an appliance for moving the patient's teeth from a first position towards a second position for a stage of an orthodontic treatment plan or a retainer for maintaining tooth position after treatment. Dental professionals may wish to evaluate the contact between the appliance and the patient's teeth in order to determine whether the appliance is applying forces to the patient's teeth in the proper locations to move the teeth or is not contacting the teeth and not applying movement forces, such as for a retainer.

At block 630 the 3D scan of the appliance with the coloring agent may be scanned in order to generate a three-dimensional model of the surface of the tooth receiving cavities that identifies the location of the colorant on the tooth receiving cavities. The three-dimensional model may include surface topography data, such as a 3D digital model, along with a color mapping or color texture applied to the 3D model. See, for example, image 810 of an appliance with a stain 812 applied thereon.

At block 640 the oral cavity may be imaged while the patient is wearing the aligner to generate the three-dimensional model of the patient's dentition with the appliance applied. The 3D model of a patient's teeth may be a digital representation of the patient's dentition and gingiva with the appliance applied. The 3D model may include surface topography data for the patient's teeth and/or the appliance, along with a color mapping, or color texture applied to the 3D digital model. The surface topography data can be generated by directly scanning the intraoral cavity using a suitable scanning device, such as a hand-held intraoral scanner. The color data may be indicative of where the appliance is contacting the patient's teeth. For example, locations on the appliance where the dye or colorant is less visible or color more closely matches the color of the patient's teeth may indicate contact between the appliance and the patient's teeth, due to transfer of the dye, stain, etc., from the appliance to the teeth during contact. See for example, image 814 of a dentition with an appliance applied thereon having light locating 816 indicating contact and dark locations elsewhere. Locations that are darker or the dye is more prevalent may indicate locations where the appliance is not contacting the patient's teeth or contacting the patient's teeth but with less force than other locations.

At block 650 the patient's dentition and the appliance may be imaged after the appliance is removed to generate a three-dimensional model of the patient's dentition and appliance with locations having colorant transfer from the appliance to the patient's teeth. The locations where colorant transfer from the appliance to the patient's teeth may indicate locations of contact between the appliance and the patient's teeth due to transfer of the dye, stain, etc., from the appliance to the teeth during contact. See, for example, image 822 of the dentition having a dye or colorant location 824 where dye or colorant has transferred to the dentition from the appliance and image 818 of the appliance having a location 820 that is lighter due to the transfer of colorant or dye. This updated color 3D model of a patient's teeth may be similar to the initial color 3D model and may be a digital representation of the patient's teeth and can include surface topography data, such as a 3D digital model, of the patient's intraoral cavity along with a color mapping or color texture applied to the 3D digital model. The surface topography data can be generated by directly scanning the intraoral cavity using a suitable scanning device, such as a hand-held intraoral scanner.

In some embodiments, an updated color 3D model of the appliance may be generated. In the updated color 3D model of the appliance, locations where colorant has been removed may indicate locations of contact between the appliance and the patient's teeth.

At block 660 the initial image of the patient's teeth generated at block 610 may be compared to the image of the dentition after removal of the appliance generated at block 650. The comparison may include determining the locations where colorant has been added to the patient's teeth by the wearing of the appliance. The determination may be generated based on image processing techniques, such as edge detection to locate the edges of colorant spots on the teeth or by other image processing analysis to identify differences in color between the initial image and the dyed image.

In some embodiments, the initial image of the appliance generated at block 630 may be compared with the imaging of the appliance after removal from the patient's teeth generated at block 650. The comparison may include determining the locations where colorant has been removed from the appliance and transferred to the patient's teeth by the wearing of the appliance. The determination may be generated based on image processing techniques, such as edge detection to locate the edges of locations where colorant was removed from the appliance or by other image processing analysis to identify differences in color between the initial image and an image of the appliance after being worn by the patient.

At block 670 the contact locations identified through the staining and scanning discussed at block 610 through 660 may be compared with expected locations of contact between the appliance and the teeth, such as those generated through an orthodontic treatment planning process designed to move the patient's teeth from a first position towards a second position.

In some embodiments, if the locations of contact are not as expected, then the geometry of the orthodontic appliance may be modified in order to provide the desired contact between the orthodontic appliance and the patient's teeth.

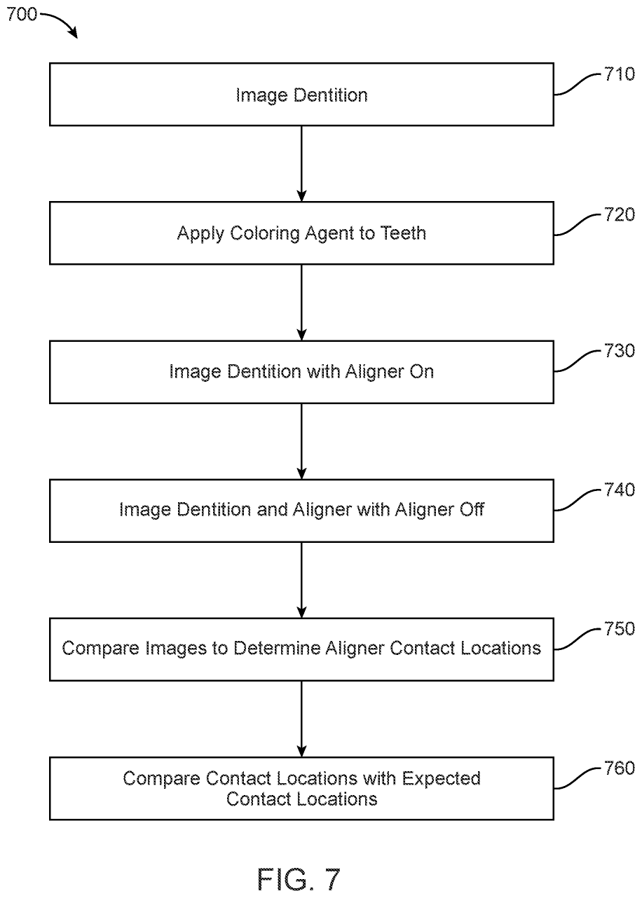

FIG. 7 shows a method 700 of using stain to detect contact between an orthodontic appliance, such as a clear aligner or retainer and teeth, in accordance with some embodiments.

At block 710 colorant or a coloring agent, such as, a stain, dye, or color paste may be added to the surfaces of the patient's teeth. The colorant may be evenly applied to the patient's teeth. The colorant may come in the form of an oral tablet that is chewed and swished around in the patient's teeth. During this process the colorant is attached to the surfaces of the patient's teeth. Colorants may be colored with a dye in the visible light spectrum, such as a red, blue, or purple. In some embodiments, colorants may be colored with dyes visible in the near infrared or ultraviolet wavelengths of light. In some embodiments, the colorants may fluoresce when exposed to certain wavelengths of light.

At block 720 a 3D scan of the of the patient's teeth with the coloring agent applied may be generated in order to create a three-dimensional model of the surface of the patient's teeth that identifies the location of the colorant on the teeth. The three-dimensional model may include surface topography data, such as a 3D digital model, along with a color mapping or color texture applied to the 3D model. See for example, image 830 of the dentition with the colorant 832 applied thereon.

At block 730 the patient's dentition may be imaged after the appliance is removed to generate a three-dimensional model of the patient's dentition with locations having colorant transferred from the patient's teeth to the appliance. The locations where colorant transferred from the patient's teeth to the appliance may indicate locations of contact between the appliance and the patient's teeth. See, for example, image 842 of the dentition having a light location 844 where dye or colorant has transferred from the dentition to the appliance and image 838 of the appliance having a location 840 that is darker due to the transfer of colorant or dye. This updated color 3D model of a patient's teeth may be similar to the initial color 3D model and may be a digital representation of the patient's teeth and can include surface topography data, such as a 3D digital model, for the patient's intraoral cavity along with a color mapping or color texture applied to the 3D digital model. The surface topography data can be generated by directly scanning the intraoral cavity using a suitable scanning device, such as a hand-held intraoral scanner.

In some embodiments, the oral cavity may be imaged while the patient is wearing the aligner to generate the three-dimensional model of the patient's dentition with the appliance applied. The 3D model of a patient's teeth may be a digital representation of the patient's dentition and gingiva with the appliance applied. The 3D model may include surface topography data for the patient's teeth and/or the appliance, along with a color mapping, or color texture applied to the 3D digital model. The surface topography data can be generated by directly scanning the intraoral cavity using a suitable scanning device, such as a hand-held intraoral scanner. The color data may be indicative of where the appliance is contacting the patient's teeth. For example, locations on the appliance where the dye or colorant is less visible or color more closely matches the color of the patient's teeth may indicate contact between the appliance and the patient's teeth, due to transfer of the dye, stain, etc., from the appliance to the teeth during contact. See for example, image 834 of a dentition with an appliance applied thereon having light locating 836 indicating contact and dark locations elsewhere. Locations that are darker or the dye is more prevalent may indicate locations where the appliance is not contacting the patient's teeth or contacting the patient's teeth but with less force than other locations.

In some embodiments, a color 3D model of the appliance may be generated. In the color 3D model of the appliance, locations where colorant has been added may indicate locations of contact between the appliance and the patient's teeth.

At block 740 the images of the dentition with the coloring agent on before applying the appliance, such as those generated at block 720, may be compared with images of the dentition after the appliance has been applied and removed from the patient's teeth, such as those generated at block 730.

The comparison may include determining the locations where colorant has been removed from the patient's teeth by the wearing of the appliance. The determination may be generated based on image processing techniques, such as edge detection to locate the edges of colorant spots on the teeth or by other image processing analysis to identify differences in color between the initial image of the dyed teeth and the image of the teeth after wearing the appliance.

In some embodiments, the image of the appliance after removal generated at block 730 may be analyzed. The analysis may include determining the locations where colorant has been added to the appliance and transferred from the patient's teeth by the wearing of the appliance. The determination may be generated based on image processing techniques, such as edge detection to locate the edges of locations where colorant was added to the appliance.

At block 750 the contact locations identified through the staining and scanning discussed at block 710 through 740 may be compared with expected locations of contact between the appliance and the teeth, such as those generated through an orthodontic treatment planning process designed to move the patient's teeth from a first position towards a second position.

In some embodiments, if the locations of contact are not as expected then the geometry of the orthodontic appliance may be modified in order to provide the desired contact between the orthodontic appliance and the patient's teeth.

### Computing System

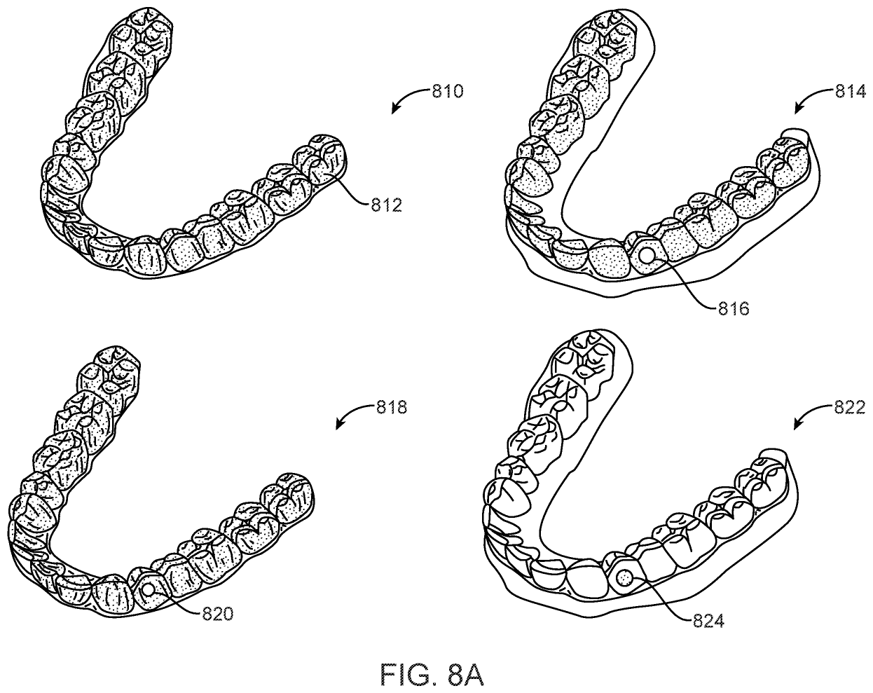

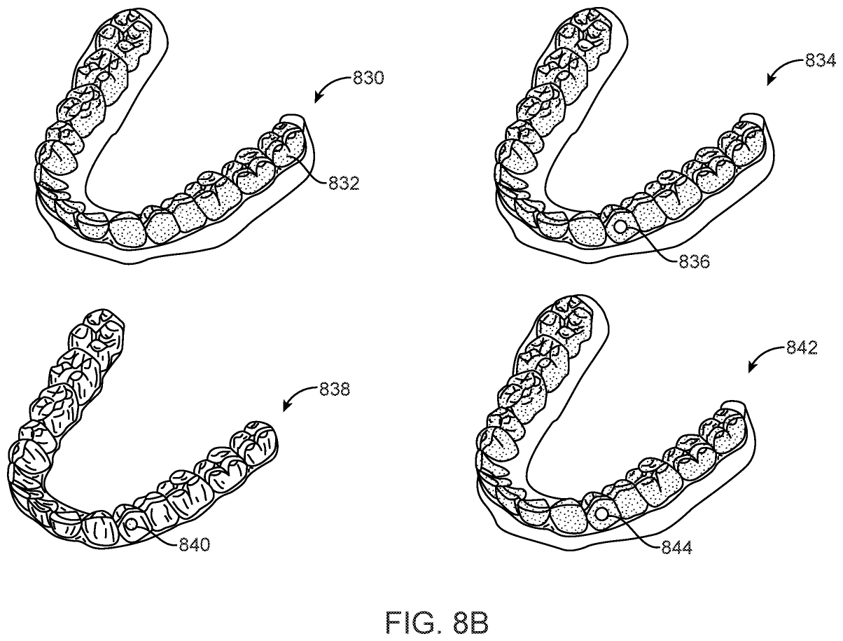

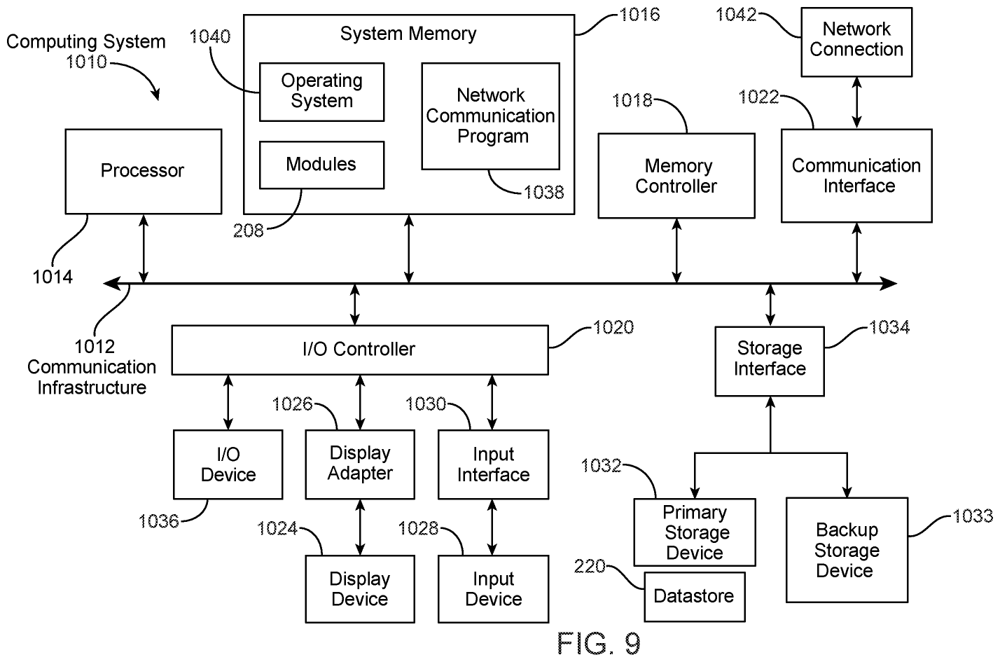

FIG. 9 is a block diagram of an example computing system 1010 capable of implementing one or more of the embodiments described and/or illustrated herein. For example, all or a portion of computing system 1010 may perform and/or be a means for performing, either alone or in combination with other elements, one or more of the steps described herein (such as one or more of the steps illustrated in FIGS. 1-8, and 11-14). All or a portion of computing system 1010 may also perform and/or be a means for performing any other steps, methods, or processes described and/or illustrated herein.

Computing system 1010 broadly represents any single or multi-processor computing device or system capable of executing computer-readable instructions. Examples of computing system 1010 include, without limitation, workstations, laptops, client-side terminals, servers, distributed computing systems, handheld devices, or any other computing system or device. In its most basic configuration, computing system 1010 may include at least one processor 1014 and a system memory 1016.

Processor 1014 generally represents any type or form of physical processing unit (e.g., a hardware-implemented central processing unit) capable of processing data or interpreting and executing instructions. In certain embodiments, processor 1014 may receive instructions from a software application or module. These instructions may cause processor 1014 to perform the functions of one or more of the example embodiments described and/or illustrated herein.

System memory 1016 generally represents any type or form of volatile or non-volatile storage device or medium capable of storing data and/or other computer-readable instructions. Examples of system memory 1016 include, without limitation, Random Access Memory (RAM), Read Only Memory (ROM), flash memory, or any other suitable memory device. Although not required, in certain embodiments computing system 1010 may include both a volatile memory unit (such as, for example, system memory 1016) and a non-volatile storage device (such as, for example, primary storage device 1032, as described in detail below). In one example, software, such as instructions for execution by a processor for carrying out the methods of any of FIGS. 1-8, and 11-14, may be loaded into system memory 1016.

In some examples, system memory 1016 may store and/or load an operating system 1040 for execution by processor 1014. In one example, operating system 1040 may include and/or represent software that manages computer hardware and software resources and/or provides common services to computer programs and/or applications on computing system 1010. Examples of operating system 1040 include, without limitation, LINUX, JUNOS, MICROSOFT WINDOWS, WINDOWS MOBILE, MAC OS, APPLE'S IOS, UNIX, GOOGLE CHROME OS, GOOGLE'S ANDROID, SOLARIS, variations of one or more of the same, and/or any other suitable operating system.

In certain embodiments, example computing system 1010 may also include one or more components or elements in addition to processor 1014 and system memory 1016. For example, as illustrated in FIG. 9, computing system 1010 may include a memory controller 1018, an Input/Output (I/O) controller 1020, and a communication interface 1022, each of which may be interconnected via a communication infrastructure 1012. Communication infrastructure 1012 generally represents any type or form of infrastructure capable of facilitating communication between one or more components of a computing device. Examples of communication infrastructure 1012 include, without limitation, a communication bus (such as an Industry Standard Architecture (ISA), Peripheral Component Interconnect (PCI), PCI Express (PCIe), or similar bus) and a network.

Memory controller 1018 generally represents any type or form of device capable of handling memory or data or controlling communication between one or more components of computing system 1010. For example, in certain embodiments memory controller 1018 may control communication between processor 1014, system memory 1016, and I/O controller 1020 via communication infrastructure 1012.

I/O controller 1020 generally represents any type or form of module capable of coordinating and/or controlling the input and output functions of a computing device. For example, in certain embodiments I/O controller 1020 may control or facilitate transfer of data between one or more elements of computing system 1010, such as processor 1014, system memory 1016, communication interface 1022, display adapter 1026, input interface 1030, and storage interface 1034.

As illustrated in FIG. 9, computing system 1010 may also include at least one display device 1024 coupled to I/O controller 1020 via a display adapter 1026. Display device 1024 generally represents any type or form of device capable of visually displaying information forwarded by display adapter 1026. Similarly, display adapter 1026 generally represents any type or form of device configured to forward graphics, text, and other data from communication infrastructure 1012 (or from a frame buffer, as known in the art) for display on display device 1024.

As illustrated in FIG. 9, example computing system 1010 may also include at least one input device 1028 coupled to I/O controller 1020 via an input interface 1030. Input device 1028 generally represents any type or form of input device capable of providing input, either computer or human generated, to example computing system 1010. Examples of input device 1028 include, without limitation, a keyboard, a pointing device, a speech recognition device, variations or combinations of one or more of the same, and/or any other input device.

Additionally or alternatively, example computing system 1010 may include additional I/O devices. For example, example computing system 1010 may include I/O device 1036. In this example, I/O device 1036 may include and/or represent a user interface that facilitates human interaction with computing system 1010. Examples of I/O device 1036 include, without limitation, a computer mouse, a keyboard, a monitor, a printer, a modem, a camera, a scanner, a microphone, a touchscreen device, variations or combinations of one or more of the same, and/or any other I/O device.

Communication interface 1022 broadly represents any type or form of communication device or adapter capable of facilitating communication between example computing system 1010 and one or more additional devices. For example, in certain embodiments communication interface 1022 may facilitate communication between computing system 1010 and a private or public network including additional computing systems. Examples of communication interface 1022 include, without limitation, a wired network interface (such as a network interface card), a wireless network interface (such as a wireless network interface card), a modem, and any other suitable interface. In at least one embodiment, communication interface 1022 may provide a direct connection to a remote server via a direct link to a network, such as the Internet. Communication interface 1022 may also indirectly provide such a connection through, for example, a local area network (such as an Ethernet network), a personal area network, a telephone or cable network, a cellular telephone connection, a satellite data connection, or any other suitable connection.

In certain embodiments, communication interface 1022 may also represent a host adapter configured to facilitate communication between computing system 1010 and one or more additional network or storage devices via an external bus or communications channel. Examples of host adapters include, without limitation, Small Computer System Interface (SCSI) host adapters, Universal Serial Bus (USB) host adapters, Institute of Electrical and Electronics Engineers (IEEE) 1394 host adapters, Advanced Technology Attachment (ATA), Parallel ATA (PATA), Serial ATA (SATA), and External SATA (eSATA) host adapters, Fibre Channel interface adapters, Ethernet adapters, or the like. Communication interface 1022 may also allow computing system 1010 to engage in distributed or remote computing. For example, communication interface 1022 may receive instructions from a remote device or send instructions to a remote device for execution.

In some examples, system memory 1016 may store and/or load a network communication program 1038 for execution by processor 1014. In one example, network communication program 1038 may include and/or represent software that enables computing system 1010 to establish a network connection 1042 with another computing system (not illustrated in FIG. 9) and/or communicate with the other computing system by way of communication interface 1022. In this example, network communication program 1038 may direct the flow of outgoing traffic that is sent to the other computing system via network connection 1042. Additionally or alternatively, network communication program 1038 may direct the processing of incoming traffic that is received from the other computing system via network connection 1042 in connection with processor 1014.

Although not illustrated in this way in FIG. 9, network communication program 1038 may alternatively be stored and/or loaded in communication interface 1022. For example, network communication program 1038 may include and/or represent at least a portion of software and/or firmware that is executed by a processor and/or Application Specific Integrated Circuit (ASIC) incorporated in communication interface 1022.

As illustrated in FIG. 9, example computing system 1010 may also include a primary storage device 1032 and a backup storage device 1033 coupled to communication infrastructure 1012 via a storage interface 1034. Storage devices 1032 and 1033 generally represent any type or form of storage device or medium capable of storing data and/or other computer-readable instructions. For example, storage devices 1032 and 1033 may be a magnetic disk drive (e.g., a so-called hard drive), a solid state drive, a floppy disk drive, a magnetic tape drive, an optical disk drive, a flash drive, or the like. Storage interface 1034 generally represents any type or form of interface or device for transferring data between storage devices 1032 and 1033 and other components of computing system 1010. In one example, digital models of teeth and/or images of teeth may be stored and/or loaded in primary storage device 1032.

In certain embodiments, storage devices 1032 and 1033 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 1032 and 1033 may also include other similar structures or devices for allowing computer software, data, or other computer-readable instructions to be loaded into computing system 1010. For example, storage devices 1032 and 1033 may be configured to read and write software, data, or other computer-readable information. Storage devices 1032 and 1033 may also be a part of computing system 1010 or may be a separate device accessed through other interface systems.

Many other devices or subsystems may be connected to computing system 1010. Conversely, all of the components and devices illustrated in FIG. 9 need not be present to practice the embodiments described and/or illustrated herein. The devices and subsystems referenced above may also be interconnected in different ways from that shown in FIG. 9. Computing system 1010 may also employ any number of software, firmware, and/or hardware configurations. For example, one or more of the example embodiments disclosed herein may be encoded as a computer program (also referred to as computer software, software applications, computer-readable instructions, or computer control logic) on a computer-readable medium. The term “computer-readable medium,” as used herein, generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The computer-readable medium containing the computer program may be loaded into computing system 1010. All or a portion of the computer program stored on the computer-readable medium may then be stored in system memory 1016 and/or various portions of storage devices 1032 and 1033. When executed by processor 1014, a computer program loaded into computing system 1010 may cause processor 1014 to perform and/or be a means for performing the functions of one or more of the example embodiments described and/or illustrated herein. Additionally or alternatively, one or more of the example embodiments described and/or illustrated herein may be implemented in firmware and/or hardware. For example, computing system 1010 may be configured as an Application Specific Integrated Circuit (ASIC) adapted to implement one or more of the example embodiments disclosed herein.

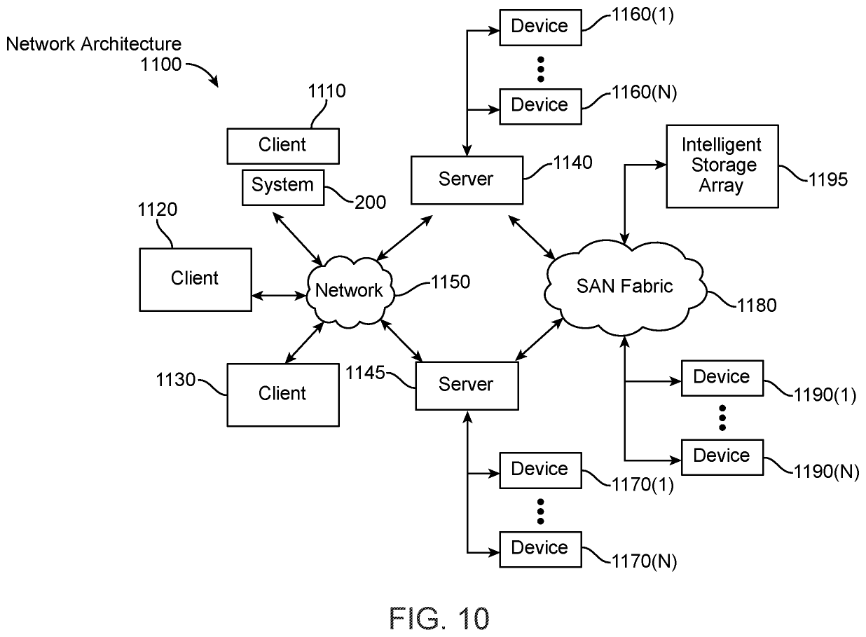

FIG. 10 is a block diagram of an example network architecture 1100 in which client systems 1110, 1120, and 1130 and servers 1140 and 1145 may be coupled to a network 1150. As detailed above, all or a portion of network architecture 1100 may perform and/or be a means for performing, either alone or in combination with other elements, one or more of the steps disclosed herein (such as one or more of the steps illustrated in FIGS. 1-8, and 11-14). All or a portion of network architecture 1100 may also be used to perform and/or be a means for performing other steps and features set forth in the instant disclosure.

Client systems 1110, 1120, and 1130 generally represent any type or form of computing device or system, such as example computing system 1010 in FIG. 9. Similarly, servers 1140 and 1145 generally represent computing devices or systems, such as application servers or database servers, configured to provide various database services and/or run certain software applications. Network 1150 generally represents any telecommunication or computer network including, for example, an intranet, a WAN, a LAN, a PAN, or the Internet.

As illustrated in FIG. 10, one or more storage devices 1160(1)-(N) may be directly attached to server 1140. Similarly, one or more storage devices 1170(1)-(N) may be directly attached to server 1145. Storage devices 1160(1)-(N) and storage devices 1170(1)-(N) generally represent any type or form of storage device or medium capable of storing data and/or other computer-readable instructions. In certain embodiments, storage devices 1160(1)-(N) and storage devices 1170(1)-(N) may represent Network-Attached Storage (NAS) devices configured to communicate with servers 1140 and 1145 using various protocols, such as Network File System (NFS), Server Message Block (SMB), or Common Internet File System (CIFS).

Servers 1140 and 1145 may also be connected to a Storage Area Network (SAN) fabric 1180. SAN fabric 1180 generally represents any type or form of computer network or architecture capable of facilitating communication between a plurality of storage devices. SAN fabric 1180 may facilitate communication between servers 1140 and 1145 and a plurality of storage devices 1190(1)-(N) and/or an intelligent storage array 1195. SAN fabric 1180 may also facilitate, via network 1150 and servers 1140 and 1145, communication between client systems 1110, 1120, and 1130 and storage devices 1190(1)-(N) and/or intelligent storage array 1195 in such a manner that devices 1190(1)-(N) and array 1195 appear as locally attached devices to client systems 1110, 1120, and 1130. As with storage devices 1160(1)-(N) and storage devices 1170(1)-(N), storage devices 1190(1)-(N) and intelligent storage array 1195 generally represent any type or form of storage device or medium capable of storing data and/or other computer-readable instructions.

In certain embodiments, and with reference to example computing system 1010 of FIG. 9, a communication interface, such as communication interface 1022 in FIG. 9, may be used to provide connectivity between each client system 1110, 1120, and 1130 and network 1150. Client systems 1110, 1120, and 1130 may be able to access information on server 1140 or 1145 using, for example, a web browser or other client software. Such software may allow client systems 1110, 1120, and 1130 to access data hosted by server 1140, server 1145, storage devices 1160(1)-(N), storage devices 1170(1)-(N), storage devices 1190(1)-(N), or intelligent storage array 1195. Although FIG. 10 depicts the use of a network (such as the Internet) for exchanging data, the embodiments described and/or illustrated herein are not limited to the Internet or any particular network-based environment.

In at least one embodiment, all or a portion of one or more of the example embodiments disclosed herein may be encoded as a computer program and loaded onto and executed by server 1140, server 1145, storage devices 1160(1)-(N), storage devices 1170(1)-(N), storage devices 1190(1)-(N), intelligent storage array 1195, or any combination thereof. All or a portion of one or more of the example embodiments disclosed herein may also be encoded as a computer program, stored in server 1140, run by server 1145, and distributed to client systems 1110, 1120, and 1130 over network 1150.

As detailed above, computing system 1010 and/or one or more components of network architecture 1100 may perform and/or be a means for performing, either alone or in combination with other elements, one or more steps of an example method for virtual care.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered example in nature since many other architectures can be implemented to achieve the same functionality.

In some examples, all or a portion of the example systems disclosed herein may represent portions of a cloud-computing or network-based environment. Cloud-computing environments may provide various services and applications via the Internet. These cloud-based services (e.g., software as a service, platform as a service, infrastructure as a service, etc.) may be accessible through a web browser or other remote interface. Various functions described herein may be provided through a remote desktop environment or any other cloud-based computing environment.

In various embodiments, all or a portion of the example systems disclosed herein may facilitate multi-tenancy within a cloud-based computing environment. In other words, the software modules described herein may configure a computing system (e.g., a server) to facilitate multi-tenancy for one or more of the functions described herein. For example, one or more of the software modules described herein may program a server to enable two or more clients (e.g., customers) to share an application that is running on the server. A server programmed in this manner may share an application, operating system, processing system, and/or storage system among multiple customers (i.e., tenants). One or more of the modules described herein may also partition data and/or configuration information of a multi-tenant application for each customer such that one customer cannot access data and/or configuration information of another customer.

According to various embodiments, all or a portion of the example systems disclosed herein may be implemented within a virtual environment. For example, the modules and/or data described herein may reside and/or execute within a virtual machine. As used herein, the term “virtual machine” generally refers to any operating system environment that is abstracted from computing hardware by a virtual machine manager (e.g., a hypervisor). Additionally or alternatively, the modules and/or data described herein may reside and/or execute within a virtualization layer. As used herein, the term “virtualization layer” generally refers to any data layer and/or application layer that overlays and/or is abstracted from an operating system environment. A virtualization layer may be managed by a software virtualization solution (e.g., a file system filter) that presents the virtualization layer as though it were part of an underlying base operating system. For example, a software virtualization solution may redirect calls that are initially directed to locations within a base file system and/or registry to locations within a virtualization layer.

In some examples, all or a portion of the example systems disclosed herein may represent portions of a mobile computing environment. Mobile computing environments may be implemented by a wide range of mobile computing devices, including mobile phones, tablet computers, e-book readers, personal digital assistants, wearable computing devices (e.g., computing devices with a head-mounted display, smartwatches, etc.), and the like. In some examples, mobile computing environments may have one or more distinct features, including, for example, reliance on battery power, presenting only one foreground application at any given time, remote management features, touchscreen features, location and movement data (e.g., provided by Global Positioning Systems, gyroscopes, accelerometers, etc.), restricted platforms that restrict modifications to system-level configurations and/or that limit the ability of third-party software to inspect the behavior of other applications, controls to restrict the installation of applications (e.g., to only originate from approved application stores), etc. Various functions described herein may be provided for a mobile computing environment and/or may interact with a mobile computing environment.

In addition, all or a portion of the example systems disclosed herein may represent portions of, interact with, consume data produced by, and/or produce data consumed by one or more systems for information management. As used herein, the term “information management” may refer to the protection, organization, and/or storage of data. Examples of systems for information management may include, without limitation, storage systems, backup systems, archival systems, replication systems, high availability systems, data search systems, virtualization systems, and the like.

In some embodiments, all or a portion of the example systems disclosed herein may represent portions of, produce data protected by, and/or communicate with one or more systems for information security. As used herein, the term “information security” may refer to the control of access to protected data. Examples of systems for information security may include, without limitation, systems providing managed security services, data loss prevention systems, identity authentication systems, access control systems, encryption systems, policy compliance systems, intrusion detection and prevention systems, electronic discovery systems, and the like.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments, one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

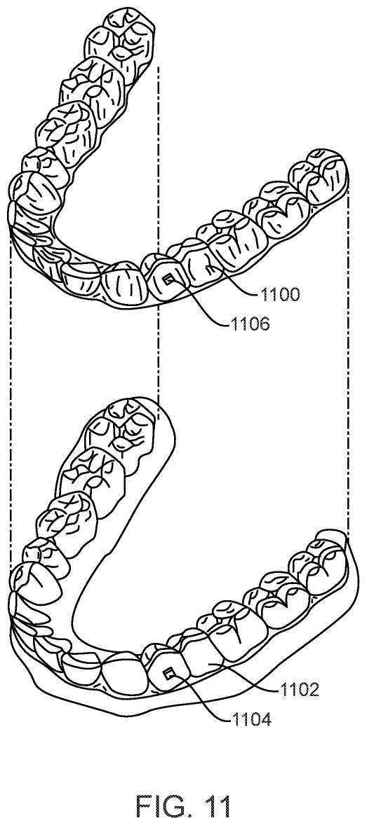

FIG. 11 illustrates an exemplary tooth repositioning appliance 1100, such as an aligner that can be worn by a patient in order to achieve an incremental repositioning of individual teeth 1102 in the jaw. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. The physical model (e.g., physical mold) of teeth can be formed through a variety of techniques, including 3D printing. The appliance can be formed by thermoforming the appliance over the physical model. In some embodiments, a physical appliance is directly fabricated, e.g., using additive manufacturing techniques, from a digital model of an appliance. In some embodiments, the physical appliance may be created through a variety of direct formation techniques, such as 3D printing. An appliance can fit over all teeth present in an upper or lower jaw, or less than all of the teeth. The appliance can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some cases, some or most, and even all, of the teeth will be repositioned at some point during treatment. Teeth that are moved can also serve as a base or anchor for holding the appliance as it is worn by the patient. In some embodiments, no wires or other means will be provided for holding an appliance in place over the teeth. In some cases, however, it may be desirable or necessary to provide individual attachments or other anchoring elements 1104 on teeth 1102 with corresponding receptacles or apertures 1106 in the appliance 1100 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the URL “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

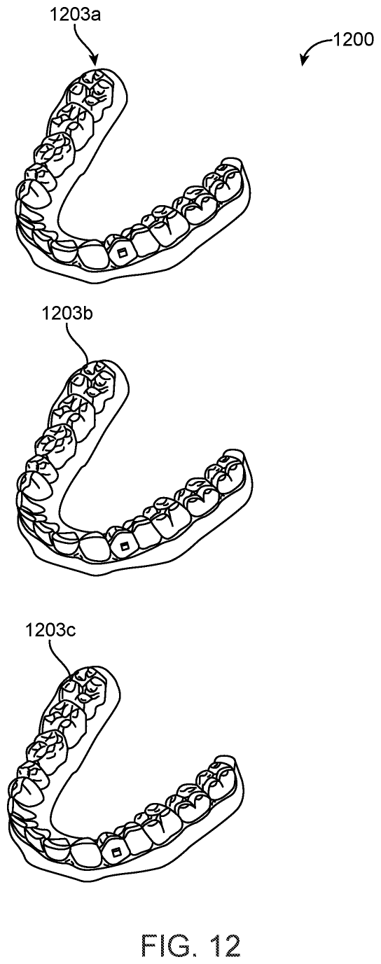

FIG. 12 illustrates a tooth repositioning system 1200 including a plurality of appliances 1203A, 1203B, 1203C. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 1200 can include a first appliance 1203A corresponding to an initial tooth arrangement, one or more intermediate appliances 1203B corresponding to one or more intermediate arrangements, and a final appliance 1203C corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

Optionally, in cases involving more complex movements or treatment plans, it may be beneficial to utilize auxiliary components (e.g., features, accessories, structures, devices, components, and the like) in conjunction with an orthodontic appliance. Examples of such accessories include but are not limited to elastics, wires, springs, bars, arch expanders, palatal expanders, twin blocks, occlusal blocks, bite ramps, mandibular advancement splints, bite plates, pontics, hooks, brackets, headgear tubes, springs, bumper tubes, palatal bars, frameworks, pin-and-tube apparatuses, buccal shields, buccinator bows, wire shields, lingual flanges and pads, lip pads or bumpers, protrusions, divots, and the like. In some embodiments, the appliances, systems and methods described herein include improved orthodontic appliances with integrally formed features that are shaped to couple to such auxiliary components, or that replace such auxiliary components.

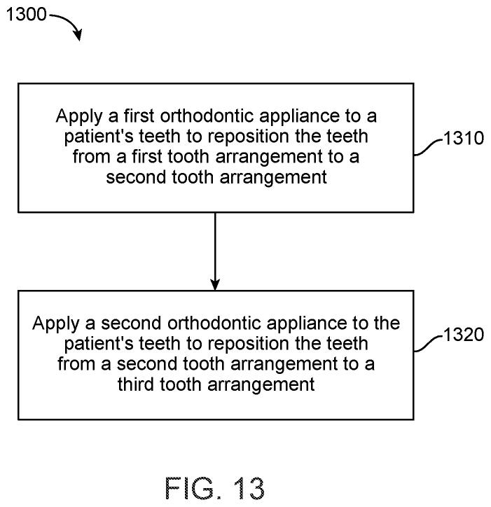

FIG. 13 illustrates a method 1300 of orthodontic treatment using a plurality of appliances, in accordance with many embodiments. The method 1300 can be practiced using any of the appliances or appliance sets described herein. In step 1310, a first orthodontic appliance is applied to a patient's teeth in order to reposition the teeth from a first tooth arrangement to a second tooth arrangement. In step 1320, a second orthodontic appliance is applied to the patient's teeth in order to reposition the teeth from the second tooth arrangement to a third tooth arrangement. The method 1300 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

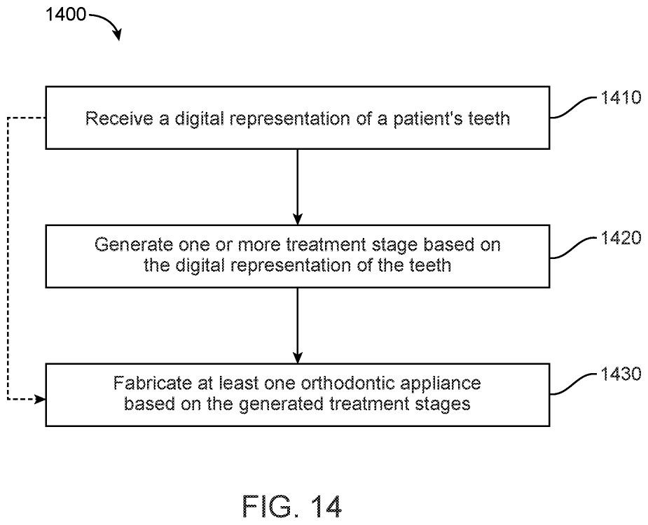

FIG. 14 illustrates a method 1400 for digitally planning an orthodontic treatment and/or design or fabrication of an appliance, in accordance with many embodiments. The method 1400 can be applied to any of the treatment procedures described herein and can be performed by any suitable data processing system. Any embodiment of the appliances described herein can be designed or fabricated using the method 1400.

In step 1410, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In step 1420, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In step 1430, at least one orthodontic appliance is fabricated based on the generated treatment stages. For example, a set of appliances can be fabricated to be sequentially worn by the patient to incrementally reposition the teeth from the initial arrangement to the target arrangement. Some of the appliances can be shaped to accommodate a tooth arrangement specified by one of the treatment stages. Alternatively or in combination, some of the appliances can be shaped to accommodate a tooth arrangement that is different from the target arrangement for the corresponding treatment stage. For example, as previously described herein, an appliance may have a geometry corresponding to an overcorrected tooth arrangement. Such an appliance may be used to ensure that a suitable amount of force is expressed on the teeth as they approach or attain their desired target positions for the treatment stage. As another example, an appliance can be designed in order to apply a specified force system on the teeth and may not have a geometry corresponding to any current or planned arrangement of the patient's teeth.

In some instances, staging of various arrangements or treatment stages may not be necessary for design and/or fabrication of an appliance. As illustrated by the dashed line in FIG. 14, design and/or fabrication of an orthodontic appliance, and perhaps a particular orthodontic treatment, may include use of a representation of the patient's teeth (e.g., receive a digital representation of the patient's teeth 1410), followed by design and/or fabrication of an orthodontic appliance based on a representation of the patient's teeth in the arrangement represented by the received representation.

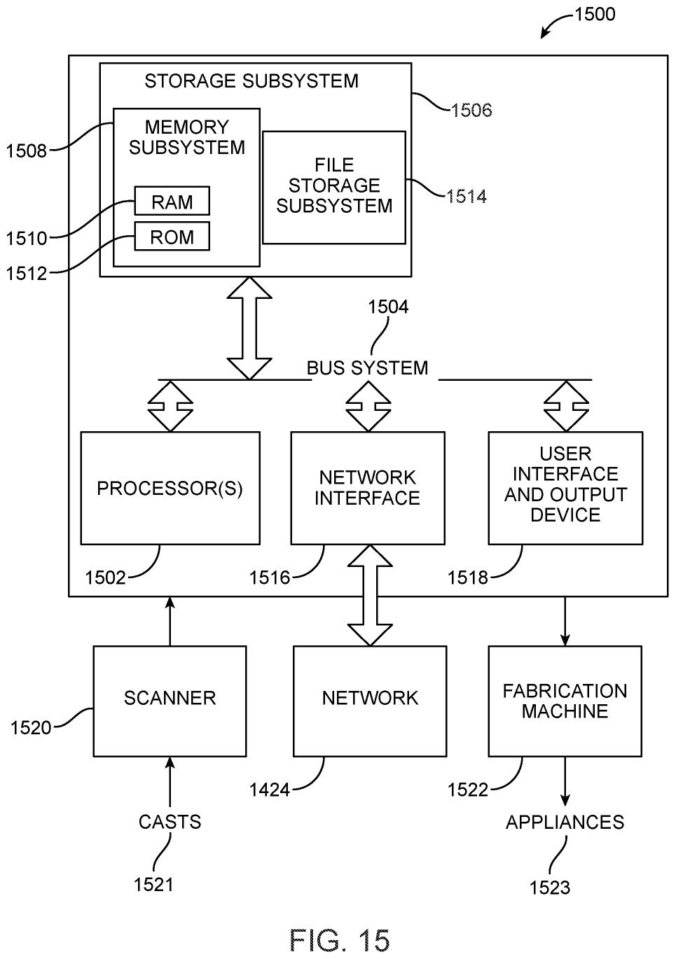

FIG. 15 is a simplified block diagram of a data processing system 1500 that may be used in executing methods and processes described herein and may incorporate aspects of the systems depicted in FIGS. 9 and 10 or may be part of the systems depicted in FIGS. 9 and 10. The data processing system 1500 typically includes at least one processor 1502 that communicates with one or more peripheral devices via bus subsystem 1504. These peripheral devices typically include a storage subsystem 1506 (memory subsystem 1508 and file storage subsystem 1514), a set of user interface input and output devices 1518, and an interface to outside networks 1516. This interface is shown schematically as “Network Interface” block 1516, and is coupled to corresponding interface devices in other data processing systems via communication network interface 1524. Data processing system 1500 can include, for example, one or more computers, such as a personal computer, workstation, mainframe, laptop, and the like.

The user interface input devices 1518 are not limited to any particular device, and can typically include, for example, a keyboard, pointing device, mouse, scanner, interactive displays, touchpad, joysticks, etc. Similarly, various user interface output devices can be employed in a system of the invention, and can include, for example, one or more of a printer, display (e.g., visual, non-visual) system/subsystem, controller, projection device, audio output, and the like.

Storage subsystem 1506 maintains the basic required programming, including computer readable media having instructions (e.g., operating instructions, etc.), and data constructs. The program modules discussed herein are typically stored in storage subsystem 1506. Storage subsystem 1506 typically includes memory subsystem 1508 and file storage subsystem 1514. Memory subsystem 1508 typically includes a number of memories (e.g., RAM 1510, ROM 1512, etc.) including computer readable memory for storage of fixed instructions, instructions and data during program execution, basic input/output system, etc. File storage subsystem 1514 provides persistent (non-volatile) storage for program and data files, and can include one or more removable or fixed drives or media, hard disk, floppy disk, CD-ROM, DVD, optical drives, and the like. One or more of the storage systems, drives, etc. may be located at a remote location, such coupled via a server on a network or via the internet/World Wide Web. In this context, the term “bus subsystem” is used generically so as to include any mechanism for letting the various components and subsystems communicate with each other as intended and can include a variety of suitable components/systems that would be known or recognized as suitable for use therein. It will be recognized that various components of the system can be, but need not necessarily be at the same physical location, but could be connected via various local-area or wide-area network media, transmission systems, etc.

Scanner 1520 includes any means for obtaining a digital representation (e.g., images, surface topography data, etc.) of a patient's teeth (e.g., by scanning physical models of the teeth such as casts 1521, by scanning impressions taken of the teeth, or by directly scanning the intraoral cavity), which can be obtained either from the patient or from treating professional, such as an orthodontist, and includes means of providing the digital representation to data processing system 1500 for further processing. Scanner 1520 may be located at a location remote with respect to other components of the system and can communicate image data and/or information to data processing system 1500, for example, via a network interface 1524. Fabrication system 1522 fabricates appliances 1523 based on a treatment plan, including data set information received from data processing system 1500. Fabrication machine 1522 can, for example, be located at a remote location and receive data set information from data processing system 1500 via network interface 1524.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters, such as numerals, refer to like elements.

The present disclosure includes the following numbered clauses.

Clause 1. A system for dental treatment, the system comprising: an intraoral scanner; non-transitory computer readable medium with instruction that, when executed by a processor, cause the system to carry out a method, the method including: generating an initial digital model of a patient's oral cavity; generating a first updated digital model of the patient's oral cavity, the first updated digital model including data representing stained locations on the patient's oral cavity; and comparing the initial digital model to the first updated digital model to identify locations of an oral problem.

Clause 2. The system of clause 1, wherein the method further comprises: generating a second updated digital model of the patient's oral cavity, the second updated digital model including data representing modified stained locations on the patient's oral cavity; and comparing the second updated digital model of the patient's oral cavity with the first updated digital model of the patient's oral cavity to determine changes in the oral problem.

Clause 3. The system of clause 2, wherein the oral problem is one or more of dental caries, plaque, or demineralization of a dentition of the patient.

Clause 4. The system of clause 2, wherein the initial digital model, the first updated digital model, and the second updated digital model, include surface topography data and color data of the patient's oral cavity.

Clause 5. The system of clause 1, wherein a stain has been applied to the teeth of the patient.

Clause 6. The system of clause 5, wherein the stain is absorbed by one or more of dental caries, plaque, or locations of demineralization of the teeth of the patient.

Clause 7. The system of clause 5, the stain on the teeth of the patient has been modified.

Clause 8. The system of clause 7, wherein the modified stain on the teeth of the patient has been modified by brushing the teeth of the patient or performing a plaque removal operation of the teeth of the patient.

Clause 9. The system of clause 8, wherein comparing the second updated digital model to the first updated digital model includes determining an amount of plaque removed from the teeth of the patient during the modification of the stain.

Clause 10. A system for dental treatment comprising: an intraoral scanner; non-transitory computer readable medium with instruction that, when executed by a processor, cause the system to carry out a method, the method including: tracking one or more oral problems of a patient's dentition by repeatedly, or a plurality of time intervals: generating an initial digital model of the patient's dentition; generating a first updated digital model of the patient's dentition, the first updated digital model including data representing stained locations on teeth of the patient; comparing the initial digital model to the first updated digital model to identify locations of an oral problem; generating diagnostic data of the oral problem based on the comparison; and determining a change in the diagnostic data over the plurality of time intervals by comparing changes in the diagnostic data of the oral problem over the plurality of time intervals.

Clause 11. The system of clause 10, wherein the oral problem is one or more of dental caries, plaque, or demineralization of the patient's dentition.

Clause 12. The system of clause 10, wherein the initial digital model and the first updated digital model, include surface topography data and color data of the patient's dentition.

Clause 13. The system of clause 11, wherein a stain has been applied to the patient's teeth before generating the first updated digital model.

Clause 14. The system of clause 13, wherein the stain is absorbed by one or more of dental caries, plaque, or locations of demineralization of the patient's teeth.

Clause 15. The system of clause 14, wherein comparing changes in the diagnostic data of the oral problem over the plurality of time intervals includes determining an increase or decrease in an extent of the oral problem of over the plurality of time intervals.

Clause 16. A system for dental treatment comprising: an intraoral scanner; non-transitory computer readable medium with instruction that, when executed by a processor, cause the system to carry out a method, the method including: generating an initial digital model of a patient's dentition; detecting a defect in the patient's dentition based on the initial digital model; generating an updated digital model of the patient's dentition, the updated digital model including data representing stained locations on teeth of the patient; comparing the initial digital model to the updated digital model to identify locations of an oral problem; generating a second updated digital model of the teeth of the patient, the second updated digital model including data representing modified stained locations on the teeth of the patient; and generating a defects model of the teeth of the patient based on the second updated digital model of the patient's dentition and the initial digital model.

Clause 17. The system of clause 16, wherein the oral problem is one or more of dental caries, plaque, or demineralization of the patient's dentition.

Clause 18. The system of clause 16, wherein the initial digital model, the updated digital model, and the second updated digital model, include surface topography data and color data of the patient's dentition.

Clause 19. The system of clause 18, wherein the method further comprises applying a stain to the teeth of the patient.

Clause 20. The system of clause 19, wherein the stain is absorbed by one or more of dental caries, plaque, or locations of demineralization of the teeth of the patient.

Clause 21. The system of clause 19, wherein the initial digital model includes near infrared, ultraviolet, or florescence data of the defect.

Clause 22. A system of dental treatment, the system comprising: an intraoral scanner; non-transitory computer readable medium with instruction that, when executed by a processor, cause the system to carry out a method, the method including: generating initial digital models of a plurality of dentitions; generating updated digital models of the dentitions, the updated digital models including data representing stained locations of oral problems on the dentitions; training a machine learning algorithm based on the initial digital models and the updated digital models; generating a digital model of a patient's dentition; and detecting oral problems in the patient's dentition with the machine learning algorithm.

Clause 23. The system of clause 22, wherein the oral problem is one or more of dental caries, plaque, or demineralization of the patient's dentition.

Clause 24. The system of clause 22, wherein the digital models include surface topography data and color data of the patient's dentition.

Clause 25. The system of clause 23, wherein the plurality of dentitions are stained before generating the updated digital models of the dentitions.

Clause 26. The system of clause 25, wherein the method further comprises: tagging the initial digital models of the plurality of dentitions with oral problems based on the data representing stained locations of oral problems on the dentitions, wherein training a machine learning algorithm based on the initial digital models and the updated digital models includes training the machine learning algorithm with the tagged initial digital models.

Clause 27. A system for dental treatment, the system comprising: an intraoral scanner; non-transitory computer readable medium with instruction that, when executed by a processor, cause the system to carry out a method, the method including: generating an initial digital model of a patient's dentition; applying a coloring agent to internal surfaces of an orthodontic appliance; generating a second digital model of the patient's dentition after wearing and removing the orthodontic appliance; comparing the initial digital model of the patient's dentition with the second digital model of the patient's dentition to determine locations of contact between the orthodontic appliance and the patient's teeth.

Clause 28. The system of clause 27, wherein the method further comprises: generating an initial digital model of colored internal surfaces of the orthodontic appliance; and generating a second digital model of the orthodontic appliance after wearing and removing the orthodontic appliance.

Clause 29. The system of clause 28, wherein the comparing includes comparing the initial digital model of colored internal surfaces of the orthodontic appliance with the second digital model of colored internal surfaces of the orthodontic appliance to determine locations of contact between the orthodontic appliance and the patient's teeth.

Clause 30. The system of clause 29, wherein the method further comprises: comparing the determined contact points with expected contact points based on an orthodontic treatment plan; and modifying the geometry of the orthodontic appliance if the contact points do not match.

Clause 31. A system for dental treatment, the system comprising: an intraoral scanner; non-transitory computer readable medium with instruction that, when executed by a processor, cause the system to carry out a method, the method including: generating an initial digital model of a patient's dentition, wherein the patient's dentition has a coloring agent applied thereto; generating a second digital model of the patient's dentition after wearing and removing an orthodontic appliance; comparing the initial digital model of the patient's dentition with the second digital model of the patient's dentition to determine locations of contact between the orthodontic appliance and the patient's teeth.

Clause 32. The system of any one of clauses 31, wherein, the method further comprises: comparing the determined contact points with expected contact points based on an orthodontic treatment plan; and modifying the geometry of the orthodontic appliance if the contact points do not match.

Clause 33. A method for dental treatment comprising: generating an initial digital model of a patient's oral cavity; generating a first updated digital model of the patient's oral cavity, the first updated digital model including data representing stained locations on the patient's oral cavity; and comparing the initial digital model to the first updated digital model to identify locations of an oral problem.

Clause 34. The method of clause 33, further comprising: generating a second updated digital model of the patient's oral cavity, the second updated digital model including data representing modified stained locations on the patient's oral cavity; and comparing the second updated digital model of the patient's oral cavity with the first updated digital model of the patient's oral cavity to determine changes in the oral problem.

Clause 35. The method of clause 34, wherein the oral problem is one or more of dental caries, plaque, or demineralization of a dentition of the patient.

Clause 36. The method of clause 34, wherein the initial digital model, the first updated digital model, and the second updated digital model, include surface topography data and color data of the patient's oral cavity.

Clause 37. The method of clause 35, further comprising applying a stain to teeth of the patient.

Clause 38. The method of clause 37, wherein the stain is absorbed by one or more of dental caries, plaque, or locations of demineralization of the teeth of the patient.

Clause 39. The method of clause 37, further comprising modifying the stain on the teeth of the patient.

Clause 40. The method of clause 39, wherein modifying the stain on the teeth of the patient includes brushing the teeth of the patient or performing a plaque removal operation of the teeth of the patient.

Clause 41. The method of clause 40, wherein comparing the second updated digital model to the first updated digital model includes determining an amount of plaque removed from the teeth of the patient during the modification of the stain.

Clause 42. A method for dental treatment comprising: tracking one or more oral problems of a patient's dentition by repeatedly, or a plurality of time intervals: generating an initial digital model of the patient's dentition; generating a first updated digital model of the patient's dentition, the first updated digital model including data representing stained locations on teeth of the patient; comparing the initial digital model to the first updated digital model to identify locations of an oral problem; generating diagnostic data of the oral problem based on the comparison; and determining a change in the diagnostic data over the plurality of time intervals by comparing changes in the diagnostic data of the oral problem over the plurality of time intervals.

Clause 43. The method of clause 42, wherein the oral problem is one or more of dental caries, plaque, or demineralization of the patient's dentition.

Clause 44. The method of clause 42, wherein the initial digital model and the first updated digital model, include surface topography data and color data of the patient's dentition.

Clause 45. The method of clause 43, further comprising applying a stain to the patient's teeth before generating the first updated digital model.

Clause 46. The method of clause 45, wherein the stain is absorbed by one or more of dental caries, plaque, or locations of demineralization of the patient's teeth.

Clause 47. The method of clause 46, wherein comparing changes in the diagnostic data of the oral problem over the plurality of time intervals includes determining an increase or decrease in an extent of the oral problem of over the plurality of time intervals.

Clause 48. A method for dental treatment comprising: generating an initial digital model of a patient's dentition; detecting a defect in the patient's dentition based on the initial digital model; generating an updated digital model of the patient's dentition, the updated digital model including data representing stained locations on teeth of the patient; comparing the initial digital model to the updated digital model to identify locations of an oral problem; generating a second updated digital model of the teeth of the patient, the second updated digital model including data representing modified stained locations on the teeth of the patient; and generating a defects model of the teeth of the patient based on the second updated digital model of the patient's dentition and the initial digital model.

Clause 49. The method of clause 48, wherein the oral problem is one or more of dental caries, plaque, or demineralization of the patient's dentition.

Clause 50. The method of clause 48, wherein the initial digital model, the updated digital model, and the second updated digital model, include surface topography data and color data of the patient's dentition.

Clause 51. The method of clause 50, further comprising applying a stain to the teeth of the patient.

Clause 52. The method of clause 51, wherein the stain is absorbed by one or more of dental caries, plaque, or locations of demineralization of the teeth of the patient.

Clause 53. The method of clause 51, wherein the initial digital model includes near infrared, ultraviolet, or florescence data of the defect.

Clause 54. A method of dental treatment comprising: generating initial digital models of a plurality of dentitions; generating updated digital models of the dentitions, the updated digital models including data representing stained locations of oral problems on the dentitions; training a machine learning algorithm based on the initial digital models and the updated digital models; generating a digital model of a patient's dentition; and detecting oral problems in the patient's dentition with the machine learning algorithm.

Clause 55. The method of clause 54, wherein the oral problem is one or more of dental caries, plaque, or demineralization of the patient's dentition.

Clause 56. The method of clause 54, wherein the digital models include surface topography data and color data of the patient's dentition.

Clause 57. The method of clause 56, further comprising applying a stain to the plurality of dentitions before generating the updated digital models of the dentitions.

Clause 58. The method of clause 57, further comprising: tagging the initial digital models of the plurality of dentitions with oral problems based on the data representing stained locations of oral problems on the dentitions, wherein training a machine learning algorithm based on the initial digital models and the updated digital models includes training the machine learning algorithm with the tagged initial digital models.

Clause 59. A method for dental treatment comprising: generating an initial digital model of a patient's dentition; applying a coloring agent to internal surfaces of an orthodontic appliance; generating a second digital model of the patient's dentition after wearing and removing the orthodontic appliance; comparing the initial digital model of the patient's dentition with the second digital model of the patient's dentition to determine locations of contact between the orthodontic appliance and the patient's teeth.

Clause 60. The method of clause 59, further comprising: generating an initial digital model of colored internal surfaces of the orthodontic appliance; and generating a second digital model of the orthodontic appliance after wearing and removing the orthodontic appliance.

Clause 61. The method of clause 60, wherein the comparing includes comparing the initial digital model of colored internal surfaces of the orthodontic appliance with the second digital model of colored internal surfaces of the orthodontic appliance to determine locations of contact between the orthodontic appliance and the patient's teeth.

Clause 62. The method of any one of clauses 61, further comprising: comparing the determined contact points with expected contact points based on an orthodontic treatment plan; and modifying the geometry of the orthodontic appliance if the contact points do not match.

Clause 63. A method for dental treatment comprising: generating an initial digital model of a patient's dentition, wherein the patient's dentition has a coloring agent applied thereto; generating a second digital model of the patient's dentition after wearing and removing an orthodontic appliance; comparing the initial digital model of the patient's dentition with the second digital model of the patient's dentition to determine locations of contact between the orthodontic appliance and the patient's teeth.

Clause 64. The method of any one of clauses 63, further comprising: comparing the determined contact points with expected contact points based on an orthodontic treatment plan; and modifying the geometry of the orthodontic appliance if the contact points do not match.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

What is claimed is:

1. A system for dental treatment, the system comprising:
an intraoral scanner;
non-transitory computer readable medium with instruction that, when executed by a processor, cause the system to carry out a method, the method including:
generating an initial digital model of a patient's oral cavity;
generating a first updated digital model of the patient's oral cavity, the first updated digital model including data representing stained locations on the patient's oral cavity; and
comparing the initial digital model to the first updated digital model to identify locations of an oral problem.

2. The system of claim 1, wherein the method further comprises:
generating a second updated digital model of the patient's oral cavity, the second updated digital model including data representing modified stained locations on the patient's oral cavity; and
comparing the second updated digital model of the patient's oral cavity with the first updated digital model of the patient's oral cavity to determine changes in the oral problem.

3. The system of claim 2, wherein the oral problem is one or more of dental caries, plaque, or demineralization of a dentition of the patient.

4. The system of claim 2, wherein the initial digital model, the first updated digital model, and the second updated digital model, include surface topography data and color data of the patient's oral cavity.

5. The system of claim 1, wherein a stain has been applied to the teeth of the patient.

6. The system of claim 5, wherein the stain is absorbed by one or more of dental caries, plaque, or locations of demineralization of the teeth of the patient.

7. The system of claim 5, the stain on the teeth of the patient has been modified.

8. The system of claim 7, wherein the modified stain on the teeth of the patient has been modified by brushing the teeth of the patient or performing a plaque removal operation of the teeth of the patient.

9. The system of claim 8, wherein comparing the second updated digital model to the first updated digital model includes determining an amount of plaque removed from the teeth of the patient during the modification of the stain.

10. A system for dental treatment comprising:
an intraoral scanner;
non-transitory computer readable medium with instruction that, when executed by a processor, cause the system to carry out a method, the method including:
tracking one or more oral problems of a patient's dentition by repeatedly, or a plurality of time intervals:
generating an initial digital model of the patient's dentition;
generating a first updated digital model of the patient's dentition, the first updated digital model including data representing stained locations on teeth of the patient;
comparing the initial digital model to the first updated digital model to identify locations of an oral problem; and
generate diagnostic data of the oral problem based on the comparison; and
determining a change in the diagnostic data over the plurality of time intervals by comparing changes in the diagnostic data of the oral problem over the plurality of time intervals.

11. The system of claim 10, wherein the oral problem is one or more of dental caries, plaque, or demineralization of the patient's dentition.

12. The system of claim 10, wherein the initial digital model and the first updated digital model, include surface topography data and color data of the patient's dentition.

13. The system of claim 11, wherein a stain has been applied to the patient's teeth before generating the first updated digital model.

14. The system of claim 13, wherein the stain is absorbed by one or more of dental caries, plaque, or locations of demineralization of the patient's teeth.

15. The system of claim 14, wherein comparing changes in the diagnostic data of the oral problem over the plurality of time intervals includes determining an increase or decrease in an extent of the oral problem of over the plurality of time intervals.

16. A system for dental treatment comprising:
an intraoral scanner;
non-transitory computer readable medium with instruction that, when executed by a processor, cause the system to carry out a method, the method including:
generating an initial digital model of a patient's dentition;
detecting a defect in the patient's dentition based on the initial digital model;
generating an updated digital model of the patient's dentition, the updated digital model including data representing stained locations on teeth of the patient;
comparing the initial digital model to the updated digital model to identify locations of an oral problem;
generating a second updated digital model of the teeth of the patient, the second updated digital model including data representing modified stained locations on the teeth of the patient; and
generating a defects model of the teeth of the patient based on the second updated digital model of the patient's dentition and the initial digital model.

17. The system of claim 16, wherein the oral problem is one or more of dental caries, plaque, or demineralization of the patient's dentition.

18. The system of claim 16, wherein the initial digital model, the updated digital model, and the second updated digital model, include surface topography data and color data of the patient's dentition.

19. The system of claim 18, wherein the method further comprises applying a stain to the teeth of the patient.

20. The system of claim 19, wherein the stain is absorbed by one or more of dental caries, plaque, or locations of demineralization of the teeth of the patient.

21. The system of claim 19, wherein the initial digital model includes near infrared, ultraviolet, or florescence data of the defect.
